(19) United States
(12) Patent Application Publication
(10) Pub. No.: US 2009/0081681 A1
(43) Pub. Date: Mar. 26, 2009

(54) Method For Genetic Selection Of High-Plasmid Producing E. Coli Clones

(76) Inventors: Maria Celina Edmonds, Hazlet, NJ (US); Kristala Jones Prather, Milton, MA (US); Jerrell Herod, Baltimore, MD (US)

Correspondence Address: MERCK AND CO., INC, P O BOX 2000, RAHWAY, NJ, 07065-0907, US

(21) Appl. No.: 12/225,139

(22) PCT Filed: Mar. 13, 2007
(86) PCT No.: PCT/US2007/006287

§ 371 Date: Sep. 15, 2008

Related U.S. Application Data
(60) Provisional application No. 60/783,683, filed on Mar. 17, 2006.

(51) Int. Cl. C12Q 1/68

(52) U.S. Cl. 435/6

(57) ABSTRACT

The present invention relates to methods of selecting for highly productive clones of E. coli for the production of plasmid DNA comprising measuring the frequency of IS1 transposon insertional mutagenesis within either the plasmid or genomic DNA of transformed clonal subtypes. An increase in IS1 insertional mutagenesis is correlated with clonal subtypes likely to exhibit a low specific productivity. The PCR-based, genetic selection assays disclosed herein are amenable to high throughput analysis, reducing the time to identify highly productive clones capable of cultivating large quantities of plasmid DNA on an industrial scale.

### FIELD OF THE INVENTION

The present invention relates to methods for selecting a highly productive clonal subtype of a strain of E. coli harboring a plasmid DNA which comprises comparing IS1 transposition activity among clonal subtypes of the same strain, wherein those clones displaying a comparatively lower transposition activity represent potential highly productive clonal subtypes. PCR-based assays are disclosed to measure the frequency of IS1 transposon insertional mutagenesis within either plasmid or genomic DNA of transformed clonal subtypes. These genetic selection assays are amenable to high throughput analysis, reducing the amount of time to identify highly productive clonal subtypes capable of cultivating large quantities of plasmid DNA on an industrial scale.

### BACKGROUND OF THE INVENTION

The manufacture and purification of large quantities of pharmaceutical-grade plasmid DNA is crucial to the applicability of both polynucleotide vaccine and gene therapy protocols for therapeutic uses. Thus, high yield plasmid DNA production and purification processes are necessary to fully develop and exploit the advantages that both DNA vaccine and gene therapy treatment options have to offer (Shamlou, 2003, Biotechnol. Appl. Biochem. 77:207-218).

Naked DNA vaccines are easily propagated as plasmid molecules in the well-studied Gram-negative bacterium Escherichia coli (“E. coli”); however, transformation of bacteria with DNA vaccine constructs can result in a heterogeneous population of clonal subtypes with respect to plasmid content. A screening process was previously developed to help isolate from this heterogeneous population those transformed E. coli clones capable of replicating and maintaining plasmid DNA at high levels (see co-pending International Application No. PCT/US2005/002911, filed Jan. 31, 2005; published as International Publication No. WO 2005/078115 on Aug. 25, 2005). Briefly, the productivity of transformed E. coli clones in a chemically-defined medium was loosely correlated to a morphological phenotype on Columbia Blood Agar. Clones which formed white, smooth, and raised circular colonies (“White” clones) were unable to amplify plasmid DNA in a fed-batch fermentation; whereas, those which formed gray, irregularly-shaped, flat and translucent colonies (“Gray” clones) were more likely to replicate plasmid DNA to high levels. A screening protocol (hereinafter, the “High-Producer Screen”) was subsequently established to identify Gray clones stably exhibiting the desired morphology through multiple rounds of cultivation in both solid and liquid medium. Clones that were stable with respect to morphology were then examined to determine plasmid content following fed-batch cultivation in shake flasks.

While the High-Producer Screen has been successfully implemented to isolate high-producing clones for several DNA vaccine candidates, the process is quite laborious and time-consuming. Growth on solid defined medium requires a three- to five-day incubation period per round, and the need to use Blood Agar as assay plates means such cultures are “dead-end” and must be cultivated in parallel so that clones from transformant to fermentor seed are maintained in blood-free medium. Therefore, experiments were undertaken to characterize the nature of the high-producer phenomenon, with the ultimate objective of developing a more robust and faster screening protocol, as disclosed herein. By understanding the genetic basis for the high-producer phenomenon, the present invention discloses improved screening protocols to more quickly identify high-plasmid producing E. coli clonal subtypes. The observation of increased IS1 transposition in low-plasmid producing E. coli DH5 clones led to the development of a variety of PCR-based assays to measure IS1 insertional mutagenesis in both plasmid and genomic DNA of transformed E. coli clones. As described herein, clones of the same strain containing the same plasmid DNA which have a lower frequency of IS1 insertional mutagenesis are identified as potential high-plasmid producing clonal subtypes. The specific productivity of said potential highly productive clonal subtypes is then tested to determine if they indeed exhibit a high plasmid copy number per cell, at which point they are identified as high-plasmid producing clones.

### SUMMARY OF THE INVENTION

The present invention relates generally to methods for selecting a highly productive clonal subtype of a strain of E. coli harboring a plasmid DNA which comprises measuring the frequency of IS1 transposon insertional mutagenesis within either the plasmid or genomic DNA of said clonal subtypes, wherein increased IS1 insertional mutagenesis is correlated with clonal subtypes likely to exhibit a low plasmid copy number per cell (i.e., low specific productivity). Importantly, the assays described herein to measure IS1 transposition in plasmid and/or genomic DNA of bacterial clonal subtypes are amenable to high throughput analysis, thus reducing the amount of time to identify a highly productive clonal subtype for, e.g., large-scale pharmaceutical-grade plasmid DNA production.

The present invention relates to a method for selecting a highly productive clonal subtype of a strain of E. coli harboring a plasmid DNA comprising: (a) comparing IS1 transposition activity in at least two clonal subtypes of the same strain harboring the same plasmid DNA, wherein the clonal subtype that displays a comparatively lower transposition activity represents a potential highly productive clonal subtype; and, (b) testing productivity of said potential highly productive clonal subtype; wherein a highly productive clonal subtype exhibits a high plasmid copy number per cell. In one embodiment of the present invention, IS1 transposition activity of a clonal subtype is determined by measuring IS1 transposon copy number in plasmid DNA samples isolated from said clone, wherein a clonal subtype with a comparatively lower IS1 transposon copy number represents a clone that displays a comparatively lower IS1 transposition activity. In a further embodiment of the present invention, IS1 transposition activity of a clonal subtype is determined by measuring the presence or absence of IS1 transposon sequences within a predetermined IS1 insertion region of the genomic DNA of said clone, wherein a clonal subtype lacking one or more IS1 insertion sequences within said predetermined region represents a clone that displays a comparatively lower IS1 transposition activity.

The present invention further relates to a method for selecting a highly productive clonal subtype of a strain of E. coli harboring a plasmid DNA comprising: (a) isolating plasmid DNA from at least two clonal subtypes of the same strain and harboring the same plasmid DNA; (b) measuring IS1 transposon copy number in said isolated plasmid DNA samples, wherein the clonal subtype that displays a comparatively lower IS1 transposon copy number represents a potential highly productive clonal subtype; and, (c) testing productivity of said potential highly productive clonal subtype; wherein a highly productive clonal subtype exhibits a high plasmid copy number per cell. According to the present invention, highly productive clonal subtypes of a strain of E. coli, including but not limited to a DH5 strain, harboring a plasmid DNA exhibit a higher plasmid copy number per cell in comparison to non-selected, transformed E. coli subtypes of the same stain that are similarly tested. In one embodiment of the present invention, the IS1 transposon copy number in isolated plasmid DNA samples is measured using a quantitative PCR (“Q-PCR”) assay, including but not limited to a Q-PCR assay that measures the relative quantity of IS1 transposon copies based on plasmid copy number. In this embodiment, the relative quantity of IS1 transposon copies based on plasmid copy number represents the IS1 transposon copy number measured as part of the described Q-PCR assay.

In one embodiment of the present invention, a Q-PCR assay is used to measure the relative quantity of IS1 transposon copies based on plasmid copy number in an isolated plasmid DNA sample, said assay comprising anplifying a first nucleotide sequence of the plasmid DNA located within an IS1 nucleotide sequence and a second nucleotide sequence of the plasmid DNA predetermined to be free of IS1 insertions, generating an IS1/plasmid copy ratio which represents the IS1 transposon copy number of a particular E. coli clonal subtype. This Q-PCR assay can be performed in multiplex mode, simultaneously amplifying both the first and second nucleotide sequences in a single reaction tube, reducing variability. The first nucleotide sequence of the plasmid DNA located within an IS1 nucleotide sequence is amplified in the presence of a nucleic acid polymerase and a set of oligonucleotides consisting of: (i) a forward PCR primer that hybridizes to a first location of the IS1 nucleotide sequence; (ii) a reverse PCR primer that hybridizes to a second location of the IS1 nucleotide sequence downstream of the first location; and, (iii) a fluorescent probe labeled with a quencher molecule and a fluorophore which emits energy at a unique emission maxima; said probe hybridizes to a location of the IS1 nucleotide sequence between the first and second locations; wherein said nucleic acid polymerase digests the fluorescent probe during amplification to dissociate said fluorophore from said quencher molecule, and a change of fluorescence upon dissociation of the fluorophore and the quencher molecule is detected, the change of fluorescence corresponding to the occurrence of IS1 amplification. The second nucleotide of the plasmid DNA, determined to be free of IS1 insertions, is also amplified in the presence of a nucleic acid polymerase and a set of oligonucleotides consisting of: (i) a forward PCR primer that hybridizes to a first location of the second nucleotide sequence; (ii) a reverse PCR primer that hybridizes to a second location of the second nucleotide sequence downstream of the first location; and, (iii) a fluorescent probe labeled with a quencher molecule and a fluorophore which emits energy at a unique emission maxima; said probe hybridizes to a location of the second nucleotide sequence between the first and second locations; wherein said nucleic acid polymerase digests the fluorescent probe during amplification to dissociate said fluorophore from said quencher molecule, and a change of fluorescence upon dissociation of the fluorophore and the quencher molecule is detected, the change of fluorescence corresponding to the occurrence of the second nucleotide sequence amplification. In one embodiment, the second nucleotide sequence of the plasmid DNA that is amplified along with the IS1 nucleotide sequence is located within a promoter sequence of the plasmid DNA, including but not limited to a nucleotide sequence located within a CMV promoter of the plasmid DNA and, thus, generating an IS1/CMV plasmid copy ratio.

After measuring the IS1 transposon copy number in at least two bacterial clonal subtypes of the same strain harboring the same plasmid DNA, the clonal type determined as having a comparatively lower IS1 transposon copy number, as defined above, is identified as a “potential” highly productive clonal subtype. The specific productivity (i.e., plasmid copy number per cell) of said potential highly productive clonal subtype is then tested by cultivating said clonal subtype in a fermentation system, preferably a small-scale fermentation system, to determine if said identified clone is indeed highly productive (i.e., exhibiting a high plasmid copy number per cell). In one embodiment of the present invention, this small-scale fermentation system consists of a shake flask fermentation system with nutrient feeding (as described in detail in co-pending International Application No. PCT/US2005/002911, published as International Publication No. WO 2005/078115). The small-scale fermentation system will ideally mimic the fermentation regime of an intended large-scale production process for generating the desired plasmid DNA.

In one embodiment of the present invention, the forward and reverse PCR primers used to amplify IS1 transposon sequences from isolated plasmid DNA samples in the described Q-PCR assay consist of IS1-Q-F (SEQ ID NO:6) and IS1-Q-R (SEQ ID NO:7), respectively, and the fluorescent probe consists of IS1-Q-P2 (SEQ ID NO:8). In another embodiment of the present invention, the forward and reverse PCR primers used to amplify the second nucleotide sequence from isolated plasmid DNA samples in the described Q-PCR assay consist of CMV-Q-F (SEQ ID NO:3) and CMV-Q-R (SEQ ID NO:4), respectively, and the fluorescent probe consists of CMV-Q-P2 (SEQ ID NO:5). The fluorescent probes are labeled with both a fluorophore and a quencher molecule.

The present invention further relates to an IS1 quantitative PCR assay, similar to that described above, comprising indirectly calculating the predicted quantity of IS1 transposon copies contributed from residual genomic DNA present in isolated plasmid DNA samples from bacterial clonal subtypes, wherein said predicted quantity of IS1 transposon copies is subtracted from the IS1/plasmid copy number, generating a corrected IS1/plasmid copy ratio. In one embodiment of this part of the present invention, the predicted contribution of IS1 transposon copies from residual genomic DNA present in a plasmid DNA sample is indirectly measured using a second QPCR assay, wherein said assay measures the relative quantity of 23s rDNA based on plasmid copy number, generating a 23s rDNA/plasmid copy ratio. Said 23s rDNA/plasmid copy ratio is subtracted from the IS1/plasmid copy ratio to provide a corrected IS1/plasmid copy ratio. This Q-PCR assay can be performed in multiplex mode, simultaneously amplifying both a 23r DNA sequence and the same nucleotide sequence of plasmid DNA determined to be free of IS1 insertions (see supra). In one embodiment of the present invention, the forward and reverse PCR primers used to amplify a 23s rDNA sequence in the Q-PCR assay described herein consist of 23s-FID (SEQ ID NO:11) and 23s-RID (SEQ ID NO:12), respectively, and the fluorescent probe consists of 23s-Pfam (SEQ ID NO:13). In another embodiment of the present invention, the sequence predetermined to be free of IS1 insertions is contained within a CMV promoter region of the plasmid DNA, generating a 23s rDNA/CMV copy ratio which is subtracted from the IS1/CMV copy ratio to generate a corrected IS1/CMV copy ratio.

The present invention also relates to methods for selecting a highly productive clonal subtype of a strain of E. coli harboring a plasmid DNA comprising detecting the presence or absence of one or more IS1 transposon insertion sequences within a region of the bacterial genomic DNA predetermined to be an IS1 insertion region, wherein a clonal subtype lacking IS1 transposon sequences within said IS1 insertion region represents a potential highly productive clonal subtype. PCR-based assays are disclosed that can detect the presence or absence of IS1 transposon sequences inserted within the predetermined IS1 insertion region. These assays are amenable to high throughput analysis. Therefore, the present invention further relates to a method for selecting a highly productive clonal subtype of a strain of E. coli harboring a plasmid DNA comprising: (a) detecting the presence or absence of an IS1 transposon sequence within a predetermined IS1 insertion region of the genomic DNA of said clonal subtype, wherein a clonal subtype lacking an IS1 transposon sequence within said IS1 insertion region represents a potential highly productive clonal subtype; and, (b) testing productivity of said potential highly productive clonal subtype; where a highly productive clonal subtype exhibits a high plasmid copy number per cell.

In a further embodiment, a TaqMan-based Q-PCR assay is used to detect the presence or absence of IS1 insertional sequences within a region of the genomic DNA of an E. coli clonal subtype, wherein said region of the genomic DNA has been predetermined to accept IS1 insertions and spans less than about 20 contiguous nucleotides of said genomic DNA (i.e., representing an “IS1 insertion site”). The Q-PCR assay that detects the presence or absence of a specific IS1 insertion within said IS1 insertion region amplifies a portion of the genomic DNA that contains said region in the presence of a nucleic acid polymerase and a set of oligonucleotides consisting of: (i) a fluorescent probe labeled with a quencher molecule and a fluorophore which emits energy at a unique emission maxima, wherein said probe hybridizes to a location within the genomic DNA that spans the IS1 insertion region only when said genomic DNA lacks an IS1 transposon sequence within said region; (ii) a forward PCR primer that hybridizes to a location of the genomic DNA upstream of the fluorescent probe; and, (iii) a reverse PCR primer that hybridizes to a location of the genomic DNA downstream of the fluorescent probe; wherein said nucleic acid polymerase digests the fluorescent probe during amplification to dissociate said fluorophore from said quencher molecule, and a change of fluorescence upon dissociation of the fluorophore and the quencher molecule is detected, the change of fluorescence corresponding to amplification of the genomic DNA and the absence of an IS1 transposon sequence within the IS1 insertion region. This assay does not require multiplexing and can be performed using a whole cell lysate, eliminating the need for isolating genomic DNA from said clone. Those clonal subtypes that lack an IS1 transposon sequence within the S11 insertion region are identified as potential highly productive clonal subtypes and will be tested to confirm their specific productivity.

In a further embodiment of the present invention, a PCR-based assay is used to detect the presence or absence of IS1 insertional sequences within a region of the genomic DNA of an E. coli clonal subtype, wherein said region of the genomic DNA has been predetermined to accept IS1 insertions and spans greater than about 20 contiguous nucleotide of said genomic DNA (i.e., representing an “IS1 insertion hotspot”). Said PCR assay amplifies a region of the genomic DNA in the presence of a nucleic acid polymerase and a set of oligonucleotides consisting of: (i) a first PCR primer that hybridizes to a location of the genomic DNA outside of the IS1 insertion region (i.e., outside of the IS1 insertion hotspot); and, (ii) a second PCR primer that hybridizes to a location of the genomic DNA within an IS1 transposon sequence; wherein the presence of an IS1 transposon sequence within the IS1 insertion region results in exponential amplification of said portion of the genomic DNA due to hybridization of and amplification from both PCR primers. The absence of an IS1 transposon sequence within the IS1 insertion region results in linear amplification of only one strand of the genomic DNA due to hybridization of only the first PCR primer. The exponential amplification of the genomic DNA can be visually detected by identifying amplified nucleic acid fragments of approximate target size or fluorescently detected in real-time by adding a nucleic acid stain that binds to double-stranded DNA (e.g., SYBR® Green). Those clonal subtypes that lack IS1 transposon sequences within the IS1 insertion region are identified as potential highly productive clonal subtypes and will be tested to confirm their specific productivity.

The present invention further relates to a method of generating a highly productive clonal subtype of a strain of E. coli harboring a plasmid DNA comprising mutating an E. coli host strain to remove all copies of IS1 sequences from the bacterial genome prior to transformation of the bacterial strain with said plasmid DNA. The present invention further relates to a mutated E. coli host strain, including but not limited to a DH5 strain, wherein all IS1 copies have been removed, and the use of said strain for the propagation of plasmid DNA.

As used herein, the term “oligonucleotide” refers to linear oligomers of natural or modified monomers or linkages, including deoxyribonucleosides, ribonucleosides, and the like, capable of specifically binding to a target polynucleotide by way of a regular pattern of monomer-to-monomer interactions, such as Watson-Crick type base pairing. For purposes of this invention, the term oligonucleotide includes both oligonucleotide probes and oligonucleotide primers.

As used herein, the term “primer” refers to an oligonucleotide that is capable of acting as a point of initiation of synthesis along a complementary strand when placed under conditions in which synthesis of a primer extension product which is complementary to a nucleic acid strand is catalyzed. Such conditions include the presence of four different deoxyribonucleotide triphosphates and a polymerization inducing agent such as DNA polymerase or reverse transcriptase, in a suitable buffer (“buffer” includes components which are cofactors, or which affect ionic strength, pH, etc.), and at a suitable temperature. As employed herein, an oligonucleotide primer can be naturally occurring, as in a purified restriction digest, or be produced synthetically. The primer is preferably single-stranded for maximum efficiency in amplification.

As used herein, “unique,” in reference to the fluorophores of the present invention, means that each fluorophore emits energy at a differing emission maxima relative to all other fluorophores used in the particular assay. The use of fluorophores with unique emission maxima allows the simultaneous detection of the fluorescent energy emitted by each of the plurality of fluorophores used in the particular assay.

As used herein, “amplicon” refers to a specific product of a PCR reaction, which is produced by PCR amplification of a sample comprising nucleic acid in the presence of a nucleic acid polymerase and a specific pair of primers.

As used herein, “oligonucleotide set” or “set of oligonucleotides” refers to a grouping of a pair of oligonucleotide primers and an oligonucleotide probe that hybridize to a specific target nucleotide sequence. Said oligonucleotide set consists of: (a) a forward primer that hybridizes to a first location of a target DNA; (b) a reverse primer that hybridizes to a second location of the same target DNA downstream of the first location; and, (c) a fluorescent probe labeled with a fluorophore and a quencher, which hybridizes to a location of the target DNA between the primers. In other words, an oligonucleotide set consists of a set of specific PCR primers capable of initiating synthesis of an amplicon specific to a specific target DNA sequence, e.g., IS1 transposon sequence, and a fluorescent probe which hybridizes to the amplicon.

As used herein, “specifically hybridizes,” in reference to oligonucleotide sets, oligonucleotide primers or oligonucleotide probes, means that said oligonucleotide sets, primers or probes hybridize to a single target DNA.

As used herein, “gene” means a segment of nucleic acid involved in producing a polypeptide chain. It includes both translated sequences (coding region) and 5′ and 3′ untranslated sequences (non-coding regions), as well as intervening sequences (introns) between individual coding segments (exons).

As used herein, “fluorophore” refers to a fluorescent reporter molecule which, upon excitation with a laser, tungsten, mercury or xenon lamp, or a light emitting diode, releases energy in the form of light with a defined spectrum. Through the process of fluorescence resonance energy transfer (FRET), the light emitted from the fluorophore can excite a second molecule whose excitation spectrum overlaps the emission spectrum of the fluorophore. The transfer of emission energy of the fluorophore to another molecule quenches the emission of the fluorophore. The second molecule is known as a quencher molecule. The term “fluorophore” is used interchangeably herein with the term “fluorescent reporter.”

As used herein “quencher” or “quencher molecule” refers to a molecule that, when linked to a fluorescent probe comprising a fluorophore, is capable of accepting the energy emitted by the fluorophore, thereby quenching the emission of the fluorophore. A quencher can be fluorescent, which releases the accepted energy as light, or non-fluorescent, which releases the accepted energy as heat, and can be attached at any location along the length of the probe.

As used herein, “probe” refers to an oligonucleotide that is capable of forming a duplex structure with a sequence in a target nucleic acid, due to complementarity of at least one sequence of the probe with a sequence in the target region, or region to be detected. The term “probe” includes an oligonucleotide as described above, with or without a fluorophore and a quencher molecule attached. The term “fluorescent probe” refers to a probe comprising a fluorophore and a quencher molecule.

As used herein, “FAM” refers to the fluorophore 6-carboxy fluorescein; “JOE” refers to the fluorophore 6-carboxy-4′,5′ dichloro-2′,7′-dimethoxyfluorescein; “TET” refers to the fluorophore 5-tetrachloro fluorescein; “VIC” refers to a proprietary fluorophore developed by Applied Biosystems; and “TAMRA” refers to the fluorophore 6-carboxy-tetramethyl-rhodamine.

As used herein, “RFLP” refers to—restriction fragment length polymorphism—.

As used herein, “DCW” refers to—dry cell weight—.

As used herein, “OD₂ pellet” refers to the mass of cells that gives an OD=2 at 600 nm when re-suspended in 1 mL of solution.

### BRIEF DESCRIPTION OF THE DRAWINGS

FIGS. 1A and B show V1Jns-nef plasmid DNA isolated from LB-grown (A) or DME-P5-grown (B) cultures, NLB-1 through NLB-10 (left to right). In gel (A), each sample lane contains 1 μl plasmid DNA from a 1 ml QIAGEN prep of culture with an average OD₆₀₀ of the 10 cultures equal to 3.7. Sample NLB-10 was added to the molecular weight marker in Lane 13. In gel (13), each sample lane contains 2 μl plasmid DNA from a 1 ml QIAGEN prep of culture with an average OD₆₀₀ of the 10 cultures equal to 12.5. (A) Lanes 1, 5, 9, 13 and (B) Lanes 1, 7, 13—New England Biolabs 1 Kb DNA ladder (0.5 μl). scDNA—supercoiled DNA. Starred lanes contain visible IS1-positive bands. FIG. 1C shows MluI digestion of selected NLB samples. Lanes 2, 4, 7, 9, 12—NLB-1, NLB-3, NLB-5, NLB-7, NLB-8; undigested. Lanes 3, 5, 8, 10, 13—NLB-1, NLB-3, NLB-5, NLB-7, NLB-8; digested. Lanes 1, 6, 11, 14-New England Biolabs 1 Kb DNA ladder (0.5 μl).

FIG. 2A shows PCR amplification of IS1 from selected samples. Lane 2—LB-grown NLB-1. Lane 3—DME-P5-grown NLB-1. Lane 4—LB-grown NLB-2. Lane 5—DME-P5-grown NLB-2. Lane 6—LB-grown pUC19. Lane 7—DH5 genomic DNA. Lane 8—DH5a genomic DNA. Lanes 1,9—GibcoBRL 1 Kb Plus DNA ladder (0.5 μl). FIG. 2B shows MluI digestion of PCR reactions utilizing plasmid preps from DME-P5-grown cultures of NLB-1 and NLB-2. Lanes 2,3—NLB-1 undigested, digested. Lanes 4,5—NLB-2 undigested, digested. Lanes 1,6—GibcoBRL 1 Kb Plus DNA ladder (0.5 μl). FIGS. 2C and 2D show PCR amplification of IS1 from NLB-3 through NLB-10 (left to right) using preps from (C) DME-P5-grown or (D) LB-grown cells. Lanes 1, 6, 11—GibcoBRL 1 Kb Plus DNA ladder (0.5 μl).

FIG. 3A shows IS1 RFLP profiles of V1jns-tpa-pol clones, with restriction enzymes AflI and AgeI. Lane 1—pIS1 positive control. Lane 2—untransformed DH5 control. Lane 3—tpa-pol-HP plasmid DNA. Lane 4-tpa-pol-HP total DNA. Lane 5-tpa-pol-LP plasmid DNA. Lane 6—tpa-pol-LP total DNA. The DIG-labeled molecular weight marker is not shown here but is visible with longer exposure times. FIG. 3B shows IS1 RFLP profiles of V1Jns-tpa-nefclones, with restriction enzymes AflII and AgeI. Lane 1—pIS1 positive control. Lane 2—untransformed DH5 control. Lane 3—tpa-nef-IP plasmid DNA. Lane 4—tpa-nef-P total DNA. Lane 5—tpa-nef-LP plasmid DNA. Lane 6—tpa-nef-LP total DNA. The DIG-labeled molecular weight marker is not shown here but is visible with longer exposure times. FIG. 3C shows IS1 RFLP profiles of untransformed DH5 and V1Jns-tpa-gag clones, with restriction enzymes AflI and AgeI. Lane 1—unadapted, untransformed DH5 control. Lane 2—untransformed DH5 adapted to defined medium DME-P5. Lane 3—tpa-gag-HP working seed plasmid DNA. Lanes 4,5—tpa-gag-HP working seed total DNA. Lane 6—tpa-gag-HP laboratory seed plasmid DNA. Lanes 7,8-tpa-gag-HP laboratory seed total DNA. Lane 9-tpa-gag-LP plasmid DNA. Lanes 10,11—tpa-gag-LP total DNA. The DIG-labeled molecular weight marker is not shown here but is visible with longer exposure times

FIG. 4 shows a plasmid map of standard pnIQ3v2. Primer and probe binding sites are indicated.

FIG. 5 shows the determination of the limit of quantitation for the 23s rDNA/CMV copy ratio assay. (⋄) Ratios determined from reactions with one primer-probe set. () Ratios determined from reactions with both primer-probe sets (multiplex). Plasmid p23sTA and PCR-amplified CMV promoter fragment were used as templates to prepare copy ratios as indicated. Linearity to 1:10⁵ copy ratio establishes the limit of quantitation.

FIG. 6 shows a schematic diagram of the fimBEA operon (for sequence specifics, see GenBank Nucleotide Database Accession Number Y10902). The location of primers used to PCR-amplify various regions of the operon are indicated as P1′, P1′-Rev, P3′, P3′-Rev, P4′, and P5, primer locations chosen based on published reports (Stentebjerg-Olesen et al., 2000, FEMS Microbiol. Lett. 182:319-325). IS1 insertion was observed in the region between primers P1′ and P3′.

FIG. 7A shows a schematic diagram of TaqMan-based high-throughput screening assay for potential highly-productive bacterial clones, wherein the IS1 insertion region spans a small number of nucleotides of the genomic DNA (“IS1 insertion site”). FIG. 7B shows a schematic diagram of a PCR-based assay for potential highly-productive bacterial clones, wherein the S11 insertion region spans a large number of contiguous nucleotides of the genomic DNA (“IS1 hotspot”). In the assay shown in 7B, a second assay must also be performed utilizing an IS1-specific primer in the opposite direction, to account for both possible orientations of the insertion.

### DETAILED DESCRIPTION OF THE INVENTION

Novel methods of selecting for highly productive clones of E. coli for the production of plasmid DNA are disclosed herein. The instant inventors/applicants have correlated increased IS1 transposition to a population of low-producing bacterial clones, which information has been used to create improved screening processes incorporating genetic selection assays to identify potential high-plasmid producing E. coli clonal subtypes. Said potential highly productive clonal subtypes are then evaluated to confirm they are indeed highly productive (i.e., exhibiting a high plasmid copy number per cell). Importantly, the assays described herein as part of the novel selection processes are amenable to high throughput analysis and, thus, will reduce the amount of time required to identify highly productive clones. Ultimately, a highly productive clonal subtype of a strain of E. coli which contains a plasmid DNA, i.e., a transformed E. coli clone, is defined as having the ability to exhibit a higher plasmid copy number per cell in comparison to non-selected, transformed E. coli clonal subtypes of the same strain containing the same plasmid DNA. Said highly productive clonal subtypes can be used, for example, in the commercial scale production of plasmid DNA intended for therapeutic polynucleotide vaccine and/or gene therapy protocols. The selection methods of the present invention are exemplified herein using the DH5 strain of E. coli; however, this exemplification is not intended to limit the scope of the present invention to the genetic selection of high-plasmid producing clones solely from the E. coli DH5 strain. It will be known to one of skill in the art that alternate strains of E. coli can be furnished for use in the selection process of the present invention.

The present invention is partly derived from the prior observation that E. coli DH5 cells transformed with a number of plasmid DNA vaccine candidates display culture heterogeneity, exhibiting at least two colony phenotypes with distinct morphologies when plated on differential and/or chemically-defined agar medium. This phenomenon is described in detail in the co-pending application filed as U.S. Provisional Application No. 60/541,894 on Feb. 4, 2004, now abandoned, and corresponding to International Application No. PCT/US2005/002911, filed Jan. 31, 2005 (published as International Publication No. WO 2005/078115 on Aug. 25, 2005); incorporated by reference herein. Colony isolation and subsequent testing of each phenotype led to the discovery of specific phenotypic clonal isolates capable of increased plasmid amplification during fermentation, generating high quantities of clinical-grade plasmid DNA. This discovery led to the development of a screening process, referred to herein as the High-Producer Screen and outlined in detail in PCT/US2005/002911 (supra), wherein highly productive clonal subtypes that exhibit a high plasmid copy number per cell are identified. The High-Producer Screen comprises a first selection step wherein potential highly productive clonal subtypes of E. coli are isolated; followed by a second selection step wherein said potential highly productive clonal subtypes isolated in step one are evaluated in a fermentation system, preferably a small-scale fermentation system, to determine which clonal subtypes are indeed highly productive. Thus, the first selection step reduces the pool of possible highly productive E. coli clonal subtypes to include only those clonal variants with the highest likelihood of demonstrating an ability to generate a greater plasmid copy number per cell in comparison to non-selected transformed E. coli cells grown under similar fermentation conditions.

Bacterial clonal subtypes have been described in the scientific literature. Phenotype switching in Candida albicans occurs as a direct result of differential gene expression (Soll, D. et al., 1995, Can. J. Bot. 73:1049-1057). Two opaque-specific genes, PEP1 and OP4, and one white-specific gene, WH11, are responsible for the white to opaque phenotype switching in pathogenic Candida. While this is associated with virulence in Candida, a similar phenomenon may exist in selecting bacterial clones with superior specific productivity. Colony variants have also been identified for pathogenic strains of Neisseria meningitidis. In this case, phenotype diversity is associated with intra-strain heterogeneity of lipopolysaccharides and class-5 outer membrane proteins (Poolman, J. T. et al., 1985, J. Med. Microbiol. 19:203-209). The effects of plasmid presence on the growth and enzymatic activity of E. coli DH5 has also been described by Mason, C. A. et al. (1989, Appl. Microbiol. Biotechnol. 32:54-60), demonstrating that plasmid copy number has a direct affect on the expression of host cell enzymes involved in carbon metabolism. Thus, the generation of E. coli clonal subtypes with different growth characteristics may result from of a variety of different events, including but not limited to mutations induced by the DNA transformation process or stress imposed by cultivating the bacteria in a selectively enriched medium.

The bacterial heterogeneity previously seen in transformed E. coli DH5 cells display two major types of colonies (as described in PCT/US2005/002911; supra). It was determined that those clones with at least the potential of later being identified as high-plasmid producing clones form phenotypically graycolored colonies when plated on Columbia Blood Agar (“Gray” clones) and cultivated at 28-30° C. Said gray colonies appear irregularly-shaped, flat and translucent. In comparison, the colonies formed by the major component of the population of transformed E. coli DH5 cells are white in color when plated on Columbia Blood Agar and cultivated at 28-30° C., circular in shape, and raised with a smooth texture (“White” clones). These “White” clones were identified to be low-plasmid producing E. coli clones. The colonies formed by the Gray clones, representing potential highly productive clonal isolates, are indistinguishable from the low-plasmid producing white colonies when plated on chemically-defined agar medium. While the potential highly-productive Gray clones can be purified directly from Columbia Blood Agar plates, it is often desirable to avoid all contact between cells used in commercial fermentation processes for the production of human therapeutic products and any blood derived material. Thus, to purify the potential highly-productive clonal subtypes (Gray clones), a somewhat laborious and time-intensive duplicate plating technique was developed to ensure that the ultimate gray clonal subtype used in the final fermentation process has failed to contact any blood products (as described in PCT/US2005/002911; supra). After selecting the potential highly-productive clonal subtypes of E. coli harboring a plasmid DNA (i.e., the Gray clones), the High-Producer Screen requires the evaluation of said clonal subtypes to determine which clones identified from the first selection step indeed possess a specific productivity (i.e., plasmid copy number per cell) greater than that of non-selected E. coli cells of the same strain, transformed with the same plasmid, and grown under the same fermentation conditions. The specific productivity of non-selected E. coli cells harboring a DNA plasmid can be readily determined by calculating the average productivity of a population of clonal isolates of said bacterial strain harboring the same plasmid DNA.

One of skill in the art will recognize that many different selection strategies are available to isolate potential highly productive bacterial clones. The High-Producer Screen as described in PCT/US2005/0029111 (supra) has proven very useful in selecting high-yielding clones for the production of plasmid for several DNA vaccine programs. Thus, the correlation of a morphological phenotype to an enriched population of high-producing clones provides one mechanism for selection of such clones. While the High-Producer Screen resulted in the delivery of high-producing seed material for several DNA vaccine candidates, the instant inventors/applicants sought to investigate the reasons behind the appearance of the heterogeneous transformant population in attempts to both further characterize and possibly improve the screening process. To this end, one early observation by the instant inventors/applicants was that the transformation efficiency of E. coli DH5 cells, i.e., the total number of recovered, plasmid-containing cells, was up to three orders of magnitude lower in defined medium than in complex broth. Consequently, an experiment was conducted in which DH5 host cells were made electro-competent and transformed with a DNA vaccine plasmid in complex medium, then shifted to defined medium, in efforts to transform and recover the cells in a manner that maximized the yield of successful transformants. However, following re-adaptation to and extended growth in defined medium, a fraction of the extracted plasmid DNA from several clones was found to contain the E. coli transposon sequence IS1. When cultured in a small-scale fermentation system (e.g., a shake flask with nutrient feeding (“SFF”) system, described in detail in PCT/US2005/002911; supra) to examine plasmid DNA content, the clones with increased IS1 transposition were all characterized as low-producers (similar to the White clones described above). Thus, there appears to be a correlation between increased IS1 transposition and a population of low-producing clones. This information was used to create the alternative screening protocols of the present invention which incorporate the genetic selection of potential high-plasmid producing E. coli clones.

The present invention relates to methods for selecting a highly productive clonal subtype of a strain of E. coli harboring a plasmid DNA which comprises comparing IS1 transposition activity among clonal subtypes of the same strain harboring the same plasmid DNA, wherein clonal subtypes displaying comparatively lower transposition activities represent potential highly productive clonal subtypes. A comparatively lower transposition activity can be readily determined by calculating the average transposition activity of a population of clonal isolates of said bacterial strain harboring the same plasmid DNA, wherein those clonal subtypes determined to have a transposition activity lower than said average are identified as exhibiting a comparatively lower transposition activity. Thus, the present invention relates to a method for selecting a highly productive clonal subtype of a strain of E. coli harboring a plasmid DNA comprising: (a) comparing IS1 transposition activity in at least two clonal subtypes of the same strain harboring the same plasmid DNA, wherein the clonal subtype that displays a comparatively lower transposition activity represents a potential highly productive clonal subtype; and, (b) testing productivity of said potential highly productive clonal subtype; wherein a highly productive clonal subtype exhibits a high plasmid copy number per cell.

In one embodiment of this portion of the present invention, IS1 transposition activity is determined by measuring IS1 transposon copy number in plasmid DNA samples isolated from clonal subtypes, wherein a comparatively lower IS1 transposon copy number indicates a comparatively lower IS1 transposition activity. Clones having a comparatively lower IS1 transposon copy number can be readily determined by calculating the average IS1 transposon copy number of a population of clonal isolates of said bacterial strain harboring the same plasmid DNA, wherein those clonal subtypes determined to have an IS1 transposon copy number lower than said average are identified as exhibiting a comparatively lower IS1 transposon copy number. In a further embodiment of the present invention, IS1 transposition activity is determined by measuring the presence or absence of one or more S1 transposon sequences within a predetermined IS1 insertion region of genomic DNA of said clonal subtypes; wherein the absence of an IS1 insertion sequence indicates a comparatively lower IS1 transposition activity. A process for pinpointing a specific location within the genomic DNA of a clonal subtype which accepts IS1 sequence insertions is described in detail in Example 5, infra.

One embodiment of the present invention relates to a method for selecting a highly productive clonal subtype of a strain of E. coli harboring a plasmid DNA comprising measuring the relative amount of IS1 transposon insertional mutagenesis in the plasmid DNA of said clonal type, wherein a low amount of IS1 transposon insertional mutagenesis is indicative of a potential highly productive clonal subtype. Thus, one embodiment of the present invention relates to a method for selecting a highly productive clonal subtype of a strain of E. coli harboring a plasmid DNA comprising: (a) isolating plasmid DNA from at least two clonal subtypes of the same strain and harboring the same plasmid DNA; (b) measuring IS1 transposon copy number in said isolated plasmid DNA samples, wherein the clonal subtype that displays a comparatively lower IS1 transposon copy number represents a potential highly productive clonal subtype; and, (c) testing productivity of said potential highly productive clonal subtype; wherein a highly productive clonal subtype exhibits a high plasmid copy number per cell. A comparatively lower IS1 transposon copy number can be readily determined by calculating the average IS1 transposon copy number of the population of clonal isolates examined, wherein those clonal subtypes determined to have a plasmid IS1 transposon copy number lower than said average value are identified as exhibiting a comparatively lower IS1 transposon copy number. Alternatively, if the IS1 transposon copy number of plasmid DNA samples from only two clonal subtypes is examined, the clone with the lowest IS1 transposon copy number represents the clonal subtype displaying the comparatively lower IS1 transposon copy number.

IS1 is a 768 base-pair transposable element known to be the smallest of the bacterial insertion sequences (Ohtsubo and Sekine, Transposable Elements, Ed. H. Saedler and A. Gierl, Berlin: Springer, 1996, 1-26). Examples of IS1 transposon sequences can be found in the NCBI GenBank Nucleotide Database under accession nos. X52534, X52537 and U49270 (IS1A/IS1E); X17345 and X52535 (IS1B/IS1C); X52536 (IS1D); X52538 (IS1F); and, V00609 (a clean copy of IS1 with no surrounding sequences). IS1 is found naturally in E. coli genomes at copy numbers up to 10, with 6 to 8 copies identified in wild-type K-12 strains (Deonier, Escherichia coli and Salmonella typhimurium: Cellular and Molecular Biology, Ed. F. Neidhardt, Washington D.C.: American Society for Microbiology, 1987, 2:982-989). Upon transposition, a 9-bp duplication of the target sequence is usually generated at the site of insertion (Ohtsubo and Sekine, 1996, supra). While IS1s found in E. coli can be grouped into four types, only the IS1A/IS1E type (see Genbank accession nos. X52534, X52537 and U49270) has been shown to transpose from chromosomal to plasmid DNA (Chen and Yeh, 1997, FEMS Microbiol. Lett. 36:275-280). IS1 causes spontaneous insertion mutations with much higher frequency than other insertion sequences (Ohtsubo and Sekine, 1996, supra) and has been identified as the causative agent for mutations in both plasmid and chromosomal DNA. For example, such mutations have been shown to suppress (fully or partially) expression of toxic or stress-inducing genes (Nakamura and Inouye, 1981, Mol. Gen. Genet. 183:107-114; Nakahama et al., 1986, Appl. Microbiol. Biotechnol. 25:262-266; and, Toba-Minowa, 1992, Gene 121:25-33); activate gene expression by disruption of regulators or distal transcriptional read-through (Hall, 1998, Mol. Biol. Evol. 15:1-5; and, Kobayashi et al., 2001, J. Bacteriol. 183:2646-2653); increase resistance of the host cell to heavy metals (Itoh et al., 1994, J. Ferment. Bioeng. 78:466-468); and, enhance plasmid segregational stability (Chew et al., 1986, FEMS Microbiol. Lett. 36:275-280). Eleven unique sites of insertion were identified in one sample of a DNA vaccine candidate, and all of these occurred either in or within 100 base-pairs of the coding region of the neomycin resistance gene, nptII (see Example 3). It is, therefore, quite possible that IS1 transposition may be the mechanism by which genetic mutations leading to the differentiation of high- and low-producers (i.e., Gray versus White clones) arise.

The presence of transposons in plasmid DNA was first noticed as higher molecular weight bands on agarose gels (see Example 1). An end-point PCR assay was employed in which so-called “imperfect-match primers” were used in standard amplification reactions for IS1. From the 5′ end, these primers consisted of 9 bp of unmatched nucleotides followed by 7 or 9 bp of perfect match nucleotides. The use of such primers resulted in a concentration-sensitive assay in which IS1 was only amplified if the template DNA concentration was above an undetermined threshold level. These assays delivered only qualitative results. Thus, in an attempt to better characterize the behavior of certain DNA vaccine clones with respect to transposon content, a quantitative PCR (“Q-PCR”) assay was designed to measure the relative quantity of IS1 copies within DNA vaccine candidates (i.e., within the plasmid DNA itself) based on plasmid copy number. This IS1/plasmid Q-PCR assay generates an IS1/plasmid copy ratio which represents a measure of IS1 transposon copy number in isolated plasmid DNA samples from transformed E. coli clonal subtypes. The assay utilizes fluorogenic TaqMan probe technology to enable detection of specific products that accumulate during PCR. Fluorescence is emitted due to the exonuclease activity of Taq DNA polymerase that digests the fluorogenic probe, separating a reporter dye on the 5′ end from a quencher dye on the 3′ end. Over the course of the PCR run, fluorescence from the reporter dye accumulates exponentially and is monitored in real-time. The fluorescence amplitude is graphed versus cycle number, and quantitation is determined by the point at which the amplitude reaches a user-defined set point, called the threshold cycle (C_{T}). Template DNA copy number can then be interpolated from an external standard curve generated from a set of known template quantities in the sample plate, or relative copy numbers can be determined by operating in multiplex mode and incorporating quantitation of a reference template in the reaction well. A second multiplex Q-PCR assay was similarly developed to calculate the predicted amount of IS1 contributed by residual genomic DNA in plasmid preparations. This assay quantitates E. coli host-cell 23s rDNA normalized to plasmid DNA copy number, generating a 23s rDNA/plasmid copy ratio. The 23s rDNA/plasmid copy ratio can be subtracted from the IS1/plasmid copy ratio to generate a “corrected” IS/plasmid copy ratio that takes into account the likely amplification of IS1 contributed by residual genomic DNA in the isolated plasmid DNA sample, providing a more accurate reading of plasmid IS1 content. Quantitation of all targets using these assays was found to be linear at a range of 10³ to 10⁸ copies of plasmid DNA per μL, allowing detection of IS1/plasmid copy ratios in the range of 100% (1:1) to 0.001% (1:10⁵). The assays are highly sensitive, whereas results suggest that an IS1 fraction on the order of 5-10% is required to be visible on agarose gels.

Without being bound by any particular theory, it is unlikely that IS1 insertions into the plasmid DNA, especially those that primarily occur within the antibiotic resistance gene as seen with V1Jns plasmids (see Example 3), would either impact the plasmid copy number or affect the amplification properties of the clones. However, transposition of an insertion sequence into the plasmid DNA results in an increase in IS1 copy number and, conceivably, an increased ability for transposition back into the genome. This behavior was observed in the acquisition of cadmium resistance in an E. coli strain (Itoh et al., 1994; supra). In an attempt to clone the genes responsible for cadmium resistance from Pseudomonas putida, a resistant E. coli transformant was obtained that carried pBR322 with a 0.8 Kb insertion. Once the host was cured of the plasmid, cadmium resistance was retained; however, the insertion was later identified as IS1 from E. coli instead of DNA from P. putida. A chromosomal rearrangement was also identified, leading to the conclusion that transposition of IS1 from the plasmid back into the genome altered the phenotype. Thus, as described herein, and without being bound by any particular theory, transposition of IS1 from the genome to the plasmid DNA and back again may contribute to the formation of low-plasmid producing E. coli clonal subtypes.

The Q-PCR assay described above is used to indirectly measure an increase in IS1 insertional mutagenesis in bacterial clonal subtypes by quantifying the IS1 transposon copy number within the plasmid DNA contained within said clones. The observations that plasmid DNA samples with significant transposition activity were all isolated from low-plasmid producing clones and that high-plasmid producing clones contained low levels of plasmid-based IS1 support the proposed theory that low producing clones are correlated with IS1 insertional mutations. While the first selection criteria for the previously described High-Producer Screen involved the analysis of morphological phenotypes, this is only a proxy for plasmid amplification behavior. Thus, as described herein, E. coli clonal subtypes can also be characterized according to their stability with respect to IS1 transposition and their preservation of high plasmid titers. The described Q-PCR assay that measures IS1 in plasmid DNA samples provides several important advantages over both agarose gel electrophoresis and end-point PCR analysis. First, the assay is highly specific. It can easily distinguish between samples containing IS1 transposons, samples containing other transposons (e.g. IS5), and transposon-negative samples through its reliance on specially designed oligonucleotide primers and fluorescent probes. Second, the high level of sensitivity offered by the Q-PCR technology allows for the quantitation of IS1 transposition over six logs of template DNA concentration while detecting targets at concentrations at least as low as 100 copies per μL (e.g., 0.6 pg/ml for V1Jns-nej).

Therefore, the present invention relates to a selection protocol to identify highly-productive clonal subtypes of a strain of E. coli, including but not limited to a K-12 strain of E. coli, such as a DH5 strain, harboring a plasmid DNA comprising measuring the relative quantity of IS1 transposon copies (i.e., IS1 transposon copy number) in isolated plasmid DNA samples from at least two clonal subtypes of the same strain harboring the same plasmid DNA and selecting the clonal subtype that displays a comparatively lower IS1 transposon copy number; wherein a clone that displays a comparatively lower IS1 transposon copy number is identified as a potential highly productive clonal subtype. The specific productivity (i.e., plasmid copy number per cell) of the potential highly productive clonal subtype is then evaluated to determine if it is indeed a highly productive clonal subtype. It is contemplated that this Q-PCR-based genetic selection process may be used to analyze greater than two clonal subtypes of the same E. coli strain harboring the same plasmid DNA, thus generating a numerical range of IS1 transposon copy numbers. In such a case, one may chose to evaluate the specific productivity not only of the clonal subtype displaying the lowest IS1 transposon copy number but of a manageable number of clonal subtypes that fall below the average IS1 transposon copy number of the assayed clones.

The potential highly productive clonal isolates are evaluated using a fermentation system, preferably a small-scale fermentation system, the size of which will be loosely dependent upon the size of the ultimate fermentation process to be used. For example, to help identity clones that will be used in a “large-scale” plasmid DNA production process (i.e., total fermentation volumes greater than standard laboratory bioreactors which can accommodate fermentation volumes of greater than about 1000 L, and can include fermentation vessels as large as 10,000 to 100,000 L), flasks ranging from about 250 mL to about 1 L are generally used in this small-scale evaluation phase. The small-scale fermentation system should also simulate the final commercial, large-scale fermentation process. As described in detail in the co-pending application disclosing the High-Producer Screen (PCT/US2005/002911; supra), potential highly productive clonal isolates can be evaluated using a shake flask with nutrient feeding (“SFF”) fermentation system whereby each flask is supplemented with continuous nutrient feeding during fermentation. A shake flask system represents a small-scale fermentation system wherein said clonal isolates are cultivated in a baffled shake flask no larger than about 1000 mL, preferably a 250 mL baffled shake flask. In one embodiment, a highly productive clonal subtype, exhibiting a high plasmid copy number per cell, is determined to have a specific productivity of greater than or equal to about 20 μg DNA/μg DCW. An alternative measurement for assessing specific productivity, typically used for small scale fermentations, is based on an OD₂ pellet, representing the mass of cells that give an OD=2 at 600 nm when re-suspended in 1 mL of solution. Thus, in another embodiment, a highly productive clonal subtype, exhibiting a high plasmid copy number per cell, is determined to have a specific productivity of greater than or equal to about 15 μg DNA/μg OD₂ pellet.

As described above, the present invention relates to a quantitative PCR (“Q-PCR”) assay, such as a TaqMan PCR assay, used to measure the IS1 transposon copy number within plasmid DNA samples of candidate DNA vaccines. Said Q-PCR assay measures the relative quantity of IS1 transposon copies based on plasmid copy number by amplifying a first nucleotide sequence of the plasmid DNA located within the IS1 nucleotide sequence and a second nucleotide sequence of the plasmid DNA predetermined to be free of IS1 insertions, generating an IS1/plasmid copy ratio which represents the number of IS1 transposon copies (i.e., IS1 transposon copy number) in a plasmid DNA sample isolated from an E. coli clonal subtype. One of skill in the art can easily identify a location within the plasmid DNA having a low probability of accepting an IS1 insertion (see, e.g., Example 3). In one embodiment, the assay is performed under multiplex mode such that the first (i.e., IS1 sequence) and second (i.e., IS1-free sequence) nucleotide sequences are amplified in the same reaction tube, reducing variability. A 5′ exonuclease fluorogenic PCR-based assay (TaqMan PCR) is described in the art which allows detection of PCR products in real-time and eliminates the need for radioactivity. See, e.g., U.S. Pat. No. 5,538,848; and Holland et al., 1991, Proc. Natl. Acad. Sci. USA 88:7276-7280. This method utilizes a labeled probe, comprising a fluorescent reporter (fluorophore) and a quencher, that hybridizes to the target DNA between the PCR primers. Excitation of the fluorophore results in the release of a fluorescent signal by the fluorophore which is quenched by the quencher. Amplicons can be detected by the 5′ to 3′ exonuclease activity of the Taq DNA polymerase, which degrades double-stranded DNA encountered during extension of the PCR primer, thus releasing the fluorophore from the probe. Thereafter, the fluorescent signal is no longer quenched and accumulation of the fluorescent signal, which is directly correlated with the amount of target DNA, can be detected in real-time with an automated fluorometer.

Automated fluorometers for performing TaqMan PCR reactions are well known in the art and can be adapted for use in this specific assay, for example, the iCycler from Bio-Rad Laboratories (Hercules, Calif.) and the Mx4000 from Stratagene (La Jolla, Calif.). In one embodiment of the present invention, the Q-PCR assays described as part of the present invention can be performed with an ABI Prism® 7900HT Sequence Detection Instrument (Applied Biosystems, Foster City, Calif.). This instrument uses a spectrograph to separate the fluorescent emission (based on wavelength) into a predictably spaced pattern across a charged-coupled device (CCD) camera. A Sequence Detection System application of the ABI Prism® 7900HT collects the fluorescent signals from the CCD camera and applies data analysis algorithms.

Nucleic acid polymerases for use in the Q-PCR assays described as part of the present invention must possess 5′ to 3′ exonuclease activity. Several suitable polymerases are known in the art, for example, Taq (Thermus aquaticus), Thr (Thermus brockianus) and Tth (Thermus thermophilus) polymerases. Taq DNA polymerase is the preferred polymerase for use in the present invention. The 5′ to 3′ exonuclease activity is characterized by the degradation of double-stranded DNA encountered during extension of the PCR primer. A fluorescent probe annealed to the amplicon will be degraded in a similar manner, thus releasing the fluorophore from the oligonucleotide. Upon dissociation of the fluorophore and the quencher, the fluorescence emitted by the fluorophore is no longer quenched, which results in a detectable change in fluorescence. During exponential growth of the PCR product, the amplicon-specific fluorescence increases to a point at which the sequence detection application, after applying a multicomponenting algorithm to the composite spectrum, can distinguish it from the background fluorescence of non-amplifying samples. The ABI Prism® 7900HT Sequence Detection Instrument also comprises a software application, which determines the threshold cycle (C_{T}) for the samples (cycle at which this fluorescence increases above a pre-determined threshold). PCR negative samples have a C_{T} equal to the total number of cycles performed and PCR positive samples have a C_{T} less than the total number of cycles performed.

Oligonucleotide probes and primers of the present invention can be synthesized by a number of methods. See, e.g., Ozaki et al., 1992, Nucleic Acids Research 20:5205-5214; Agrawal et al., 1990, Nucleic Acids Research 18:5419-5423. For example, oligonucleotide probes can be synthesized on an automated DNA synthesizer such as the ABI 3900 DNA Synthesizer (Applied Biosystems, Foster City, Calif.). Alternative chemistries, e.g., resulting in non-natural backbone groups, such as phosphorothioate, phosphoramidate, and the like, may also be employed provided that the hybridization efficiencies of the resulting oligonucleotides are not adversely affected.

The PCR amplification step of the present invention can be performed by standard techniques well known in the art (see, e.g., Sambrook, E. F. et al., Molecular Cloning: A Laboratory Manual, 2nd edition, Cold Spring Harbor Laboratory Press (1989); U.S. Pat. No. 4,683,202; and, PCR Protocols: A Guide to Methods and Applications, Eds. Innis et al., San Diego:Academic Press, Inc. (1990); all of which are hereby incorporated by reference). PCR cycling conditions typically consist of an initial denaturation step, which can be performed by heating the PCR reaction mixture to a temperature ranging from about 80° C. to about 105° C. for times ranging from about 1 to about 10 minutes. Heat denaturation is typically followed by a number of cycles, ranging from about 20 to about 50 cycles, each cycle usually comprising an initial denaturation step, followed by a primer annealing step, and concluding with a primer extension step. Alternatively, each cycle may comprise a denaturation step at one temperature ranging from about 80° C. to about 105° C., followed by a primer annealing/extension step at a lower temperature, ranging from about 60° C. to about 75° C. Enzymatic extension of the primers by the nucleic acid polymerase, e.g., Taq polymerase, produces copies of the template that can be used as templates in subsequent cycles. “Hot start” PCR reactions may be used in conjunction with the methods of the present invention to eliminate false priming and the generation of non-specific amplicons. To this end, in a preferred embodiment of this invention, the nucleic acid polymerase is AmpliTaq Gold DNA polymerase and the PCR cycling conditions include a “hot start” PCR reaction. Said polymerase is inactive until activation, which can be accomplished by incubating the PCR reaction components at 95° C. for approximately 10 minutes prior to PCR cycling. PCR methods comprising a similar initial incubation step are known in the art as “hot start” PCR assays.

In one embodiment of the present invention, oligonucleotide probes for the TaqMan Q-PCR assays described herein range from about 15 to about 40 nucleotides in length are used. In another embodiment, the oligonucleotide probes are in the range of about 15 to about 30 nucleotides in length. In an third embodiment of the present invention, the oligonucleotide probes are in the range of about 18 to about 28 nucleotides in length. The precise sequence and length of an oligonucleotide probe of the invention depends, in part, on the nature of the target polynucleotide to which it binds. The binding location and length may be varied to achieve appropriate annealing and melting properties for a particular embodiment. The 3′ terminal nucleotide of the oligonucleotide probe is preferably blocked or rendered incapable of extension by a nucleic acid polymerase. Since the DNA polymerase can only add nucleotides to a 3′ hydroxyl and not a 3′ phosphate, such blocking is conveniently carried out by phosphorylation of the 3′ terminal nucleotide.

The fluorophores of the present invention may be attached to the probe at any location of the probe, including the 5′ end, the 3′ end or internal to either end, i.e., said fluorophore may be attached to any one of the nucleotides comprising the specific sequence of nucleotides capable of hybridizing to the target DNA that the probe was designed to detect. In one embodiment of this invention, the fluorophore is attached to a 5′ terminal nucleotide of the specific sequence of nucleotides and the quencher is attached to a 3′ terminal nucleotide of the specific sequence of nucleotides. Fluorophores used in the present invention are preferably fluorescent organic dyes derivatized for attachment to the 3′ carbon or terminal 5′ carbon of the probe via a linking moiety. Quencher molecules are also preferably organic dyes, which may or may not be fluorescent. Generally, whether the quencher molecule is fluorescent or simply releases the transferred energy from the reporter by non-radioactive decay, the absorption band of the quencher should substantially overlap the fluorescent emission band of the reporter molecule. Non-fluorescent quencher molecules that absorb energy from excited reporter molecules, but which do not release the energy radiatively, are referred as “dark quenchers” or “non-fluorescent quenchers.”

Several fluorophore-quencher pairs are described in the art. See, e.g., Pesce et al., editors, Fluorescence Spectroscopy, Marcel Dekker, New York (1971); White et al, Fluorescence Analysis: A Practical Approach, Marcel Dekker, New 20 York (1970); and the like. The literature also includes references providing exhaustive lists of fluorescent and non-fluorescent molecules and their relevant optical properties, e.g., Berlman, Handbook of Fluorescence Sprectra of Aromatic Molecules, 2nd edition, New York: Academic Press (1971). Further, there is extensive guidance in the literature for derivatizing reporter and quencher molecules for covalent attachment via common reactive groups that can be added to an oligonucleotide. See, e.g., U.S. Pat. No. 3,996,345 and U.S. Pat. No. 4,351,760. Exemplary fluorophore-quencher pairs may be selected from xanthene dyes, including fluoresceins, and rhodamine dyes. Many suitable forms of these compounds are widely available with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino 8-naphthalene sulfonate and 2-p-touidinyl-6-naphthalene sulfonate. Other dyes include 3-phenyl-7-isocyanatocoumarin, acridines, such as 9-isothiocyanatoacridine and acridine orange; N-(p-(2-benzoxazolyl)phenyl) maleimide; benzoxadiazoles, D stilbenes, pyrenes, and the like.

In one embodiment of the present invention, fluorophore and quencher molecules are selected from fluorescein and rhodamine dyes. These dyes and appropriate linking methodologies for attachment to oligonucleotides are known in the art. See, e.g., Marshall, 1975, Histochemical J. 7:299-303; and U.S. Pat. No. 5,188,934. In a preferred embodiment of this invention, the fluorophores are selected from the group consisting of: 6-carboxy-fluorescein (FAM); the Applied Biosystems proprietary fluorophore, VIC; 6-carboxy-4′,5′-dichloro-2′,7′ dimethoxyfluorescein (JOE); and, 5-tetrachloro-fluorescein (TET). In a further embodiment of this invention, the quencher molecule is fluorescent, such as 6-carboxy-tetramethyl-rhodamine (TAMRA). Preferably, commercially available linking moieties are employed that can be attached to an oligonucleotide during synthesis, e.g., available from Clontech Laboratories (Palo Alto, Calif.).

In one embodiment of the present invention, the IS1/plasmid copy ratio is determined by amplifying a first nucleotide sequence of the plasmid DNA located within the IS1 nucleotide sequence and a second nucleotide sequence of the plasmid DNA determined to be free of IS1 insertions, wherein the first and second nucleotide sequences of the plasmid DNA are individually amplified in the presence of a nucleic acid polymerase and a set of oligonucleotides. The set of oligonucleotides used to amplify the first nucleotide sequence consists of: (i) a forward PCR primer that hybridizes to a first location of the IS1 nucleotide sequence; (ii) a reverse PCR primer that hybridizes to a second location of the IS1 nucleotide sequence downstream of the first location; and, (iii) a fluorescent probe labeled with a quencher molecule and a fluorophore which emits energy at a unique emission maxima; said probe hybridizes to a location within the IS1 nucleotide sequence between the first and second locations. The set of oligonucleotides used to amplify the second nucleotide sequence consists of: (i) a forward PCR primer that hybridizes to a first location of the second nucleotide sequence; (ii) a reverse PCR primer that hybridizes to a second location of the second nucleotide sequence downstream of the first location; and (iii) a fluorescent probe labeled with a quencher molecule and a fluorophore which emits energy at a unique emission maxima; said probe hybridizes to a location within the second nucleotide sequence between the first and second locations. The nucleic acid polymerase digests the fluorescent probes during amplification to dissociate said fluorophores from said quencher molecules, and a change of fluorescence upon dissociation of the fluorophores and quencher molecules is detected, the change of fluorescence corresponding to the occurrence of amplification of the first and/or second nucleotide sequences. In a further embodiment, said first and second nucleotide sequences are simultaneously amplified in multiplex mode.

In another embodiment, the forward and reverse PCR primers capable of amplifying the nucleotide sequence of IS1 consist of IS1-Q-F (5′-AGGCTCATAAGACGCCCCA-3′; SEQ ID NO:6) and IS1-Q-R (5′-ACGGTTGTTGCGCACGTAT-3′; SEQ ID NO:7), respectively, and the fluorescent probe consists of IS1-Q-P2 (5′-CGTCGCCATAGTGCGTTCACCG-3′; SEQ ID NO:8), wherein said probe is labeled with both a fluorophore and a quencher molecule, as described above. In one embodiment, the IS1-Q-F probe is labeled at the 3′ terminus with the quencher molecule TAMRA and at the 5′ terminus with the fluorophore FAM. In a further embodiment of this part of the present invention, the nucleotide sequence of the plasmid DNA determined to be free of IS1 insertions that is amplified along with the IS1 nucleotide sequence is a promoter sequence of the plasmid DNA, including but not limited to a CMV promoter sequence (e.g., a human CMV promoter). In one embodiment, the forward and reverse PCR primers capable of amplifying the nucleotide sequence of the CMV promoter may consist of CMV-Q-F (5′-GTACGGTGGGAGGTCTATATAAGCA-3′; SEQ ID NO:3) and CMV-Q-R (5′-GGAGGTCAAAACAGCGTGGAT-3′; SEQ ID NO:4), respectively, and the fluorescent probe may consist of CMV-Q-P2 (5′-TCGTTTAGTGAACCGTCAGATCGCCTG-3′; SEQ ID NO:5), wherein said probe is labeled with both a fluorophore and a quencher molecule, as described above. In one embodiment, the CMV-Q-P2 probe is labeled with the quencher molecule TAMRA at the 3′ terminus and the fluorophore VIC at the 5′ terminus.

Thus, the present invention further relates to a method for selecting a highly productive clonal subtype of a strain of E. coli harboring a plasmid DNA comprising: (a) isolating plasmid DNA from at least two clonal subtypes of the same strain harboring the same plasmid DNA; (b) measuring the relative quantity of IS1 transposon copies based on plasmid copy number in said isolated plasmid DNA sample from a first clonal subtype using a quantitative PCR assay, wherein said assay amplifies a first nucleotide sequence of the plasmid DNA located within an IS1 nucleotide sequence and a second nucleotide sequence of the plasmid DNA determined to be free of IS1 insertions, generating an IS1/plasmid copy ratio; (c) comparing the IS1/plasmid copy ratio from the first clonal subtype to the IS1/plasmid copy ratio from at least a second clonal subtype of the same strain harboring the same plasmid DNA; (d) selecting the clone that displays a comparatively lower IS1/plasmid copy ratio, wherein the clone that displays a comparatively lower IS1/plasmid copy ratio is identified as a potential highly productive clonal subtype; and, (e) testing productivity of said potential highly productive clonal subtype; wherein a highly productive clonal subtype exhibits a high plasmid copy number per cell.

As described previously, a second multiplex Q-PCR assay was developed to calculate the predicted quantity of IS1 contributed by residual genomic DNA in plasmid DNA samples of the tested E. coli clonal subtypes, further increasing the specificity of the disclosed genetic selection process by allowing for a more precise quantitation of increases in IS1 transposition activity. In standard plasmid DNA preparations, genomic DNA is co-precipitated with denatured protein, separated from the plasmid DNA, and discarded. Thus, while plasmid DNA samples isolated from bacterial cell fermentations are predominantly comprised of plasmid DNA, a small amount of genomic DNA contamination can occur. For example, plasmid DNA purified with QIAGEN columns may contain up to 3.3% genomic DNA by weight (Vilalta et al., 2002, Analytical Biochem. 301:151-153). Although the genomic DNA contamination may be minor, the high sensitivity of the Q-PCR assay of the present invention will enable detection of IS1 transposons located within said residual genomic DNA. Since the IS1/plasmid copy ratio assay described herein cannot distinguish between IS1 located within the plasmid DNA and IS1 located within the genomic/chromosomal DNA, this second multiplex Q-PCR was developed to account for the amount of IS1 contributed by baseline residual genomic DNA. Thus, in one embodiment of the present invention, the IS1/plasmid copy ratio described above, representing the relative quantity of IS1 copies based on plasmid copy number (i.e., IS1 transposon copy number), is corrected by subtracting the predicted quantity of IS1 copies contributed from residual genomic DNA present in the plasmid DNA sample, wherein the predicted quantity of IS1 copies from residual genomic DNA present in the plasmid DNA sample is measured using a quantitative PCR assay. Since, as mentioned above, the IS1/plasmid copy ratio assay cannot distinguish between plasmid- and chromosome-based IS1, in order to correct for the likely contribution of IS1 from residual genomic DNA, the Q-PCR assay measures a component of the chromosomal DNA that is thought to be present in a similar quantity to the baseline IS1 in said chromosomal DNA. For example, between 6 to 8 copies of IS1 is present in the E. coli K-12 genome (Ohtsubo and Sekine, 1996; supra), and Southern blot experiments by the inventors/applicants have shown that IS1 copy number in DH5 cells is 6 or 7 (see Example 2). Similarly, the 23s rDNA gene is present in the E. coli genome at 7 copies per cell (Jinks-Robertson and Nomura, Escherichia coli and Salmonella typhimurium: Cellular and Molecular Biology, Ed. F. Neidhardt, Washington D.C.: American Society for Microbiology, 1987, 2:1358-1385). Thus, quantitating 23s rDNA copies in isolated plasmid DNA samples provides a good approximation of the number of copies of IS1 measured in the disclosed Q-PCR assay that arise from residual genomic DNA.

Thus, in one embodiment of the present invention, the IS1/plasmid copy ratio, as described above, is corrected by subtracting the predicted contribution of IS1 copies from residual genomic DNA present in the plasmid DNA sample, wherein the predicted contribution of IS1 copies from residual genomic DNA present in the plasmid DNA sample is measured a using a Q-PCR assay. In a further embodiment, said Q-PCR assay measures the relative quantity of 23s rDNA based on plasmid copy number by amplifying a nucleotide sequence of the genomic DNA within the 23s rDNA sequence and the same nucleotide sequence of the plasmid DNA determined to be free of IS1 insertions used to generate the IS1/plasmid copy ratio, generating a 23s rDNA/plasmid copy ratio that is subtracted from the IS1/plasmid copy ratio to provide a “corrected” IS1/plasmid copy ratio.

In another embodiment of the present invention, the 23s rDNA/plasmid copy ratio is determined by amplifying a nucleotide sequence of the genomic DNA within the 23s rDNA sequence and the same nucleotide sequence of the plasmid DNA determined to be free of IS1 insertions (i.e., the second nucleotide sequence amplified when generating the IS1/plasmid copy ratio), wherein the nucleotide sequence located within the 23s rDNA sequence and the second nucleotide sequence are individually amplified in the presence of a nucleic acid polymerase and a set of oligonucleotides. The set of oligonucleotides used to amplify the 23s rDNA nucleotide sequence consists of: (i) a forward PCR primer that hybridizes to a first location of the 23s rDNA sequence; (ii) a reverse PCR primer that hybridizes to a second location of the 23s rDNA sequence downstream of the first location; and, (iii) a fluorescent probe labeled with a quencher molecule and a fluorophore which emits energy at a unique emission maxima; said probe hybridizes to a location within the 23s rDNA sequence between the first and second locations. The set of oligonucleotides used to amplify the second nucleotide sequence consists of: (i) a forward PCR primer that hybridizes to a first location of the second nucleotide sequence; (ii) a reverse PCR primer that hybridizes to a second location of the second nucleotide sequence downstream of the first location; and (iii) a fluorescent probe labeled with a quencher molecule and a fluorophore which emits energy at a unique emission maxima; said probe hybridizes to a location within the second nucleotide sequence between the first and second locations. The nucleic acid polymerase digests the fluorescent probes during amplification to dissociate said fluorophores from said quencher molecules, and a change of fluorescence upon dissociation of the fluorophore and quencher molecules is detected, the change of fluorescence corresponding to amplification of the 23s rDNA sequence and/or the second nucleotide sequence. In a further embodiment, said 23s rDNA and second nucleotide sequences are simultaneously amplified in multiplex mode.

In a further embodiment, the forward and reverse PCR primers capable of amplifying a nucleotide sequence of 23s rDNA within E. coli genomic DNA consist of 23s-FID (5′-GAAAGGCGCGCGATACAG-3′; SEQ ID NO:11) and 23s-FID (5′-GTCCCGCCCTACTCATCGA-3′; SEQ ID NO:12), respectively, and the fluorescent probe consists of 23s-Pfam (5′-CCCCGTACACAAAAATGCACATGCTG-3′; SEQ ID NO:13), wherein said probe is labeled with both a fluorophore and a quencher molecule, as described above. In one embodiment, the 23s-Pfam probe is labeled at the 3′ terminus with the quencher molecule TAMRA and at the 5′ terminus with the fluorophore FAM. In another embodiment of this part of the present invention, the nucleotide sequence of the plasmid DNA determined to be free of IS1 insertions that is amplified along with the 23s rDNA nucleotide sequence from residual genomic DNA is a promoter sequence of the plasmid DNA, including but not limited to a CMV promoter sequence (e.g., a human CMV promoter), generating a 23s rDNA/CMV copy ratio.

The present invention further relates to a method for selecting a highly productive clonal subtype of a strain of E. coli harboring a plasmid DNA comprising: (a) isolating plasmid DNA from at least two clonal subtypes of the same strain harboring the same plasmid DNA; (b) measuring the relative quantity of IS1 transposon copies based on plasmid copy number in a first plasmid DNA sample using a quantitative PCR assay, wherein said assay amplifies a first nucleotide sequence of the plasmid DNA located within an IS l nucleotide sequence and a second nucleotide sequence of the plasmid DNA predetermined to be free of IS1 insertions, generating an IS1/plasmid copy ratio; (c) calculating the predicted quantity of IS1 transposon copies contributed from residual genomic DNA present in said first plasmid DNA sample using a quantitative PCR assay that measures the relative quantity of 23s rDNA based on plasmid copy, wherein said assay amplifies a nucleotide sequence of the genomic DNA located within the 23s rDNA sequence and the second nucleotide sequence of step (b), generating a 23s rDNA/plasmid copy ratio; (d) subtracting the 23s rDNA/plasmid copy ratio from the IS1/plasmid copy ratio to generate a corrected IS1/plasmid copy ratio; (e) comparing the corrected IS1/plasmid copy ratio from the first plasmid DNA sample to a corrected IS1/plasmid copy ratio from at least a second plasmid DNA sample; (f) selecting the clone that displays a comparatively lower corrected IS1/plasmid copy ratio, wherein the clone that displays a comparatively lower corrected IS1/plasmid copy ratio is identified as a potential highly productive clonal subtype; and, (g) testing productivity of said potential highly productive clonal subtype; wherein a highly productive clonal subtype exhibits a high plasmid copy number per cell. In one embodiment of this part of the present invention, the nucleotide sequence of the plasmid DNA determined to be free of IS1 transposon insertions is a promoter region of the plasmid DNA, including but not limited to a CMV promoter region of the plasmid and, thus, e.g., generating a IS1/CMV and/or corrected IS1/CMV copy ratio.

While the genetic selection process described in detail above encompasses assessing the degree of IS1 insertional mutagenesis in an E. coli subtype harboring a plasmid DNA by measuring IS1 transposon copy number in the plasmid DNA itself, the present invention is further drawn to methods for selecting a highly productive clonal subtype of a strain of E. coli harboring a plasmid DNA which encompasses detecting increased IS1 insertional mutagenesis in the genomic DNA of the clonal subtype. PCR-based assays, amenable to high throughput analysis, are contemplated that will detect the presence or absence of IS1 transposon sequences within a region of the genomic DNA predetermined to accept IS1 insertions (i.e., a “predetermined IS1 insertion region”). The presence or absence of an IS1 transposon insertion within this predetermined region should not be confused with the baseline IS1 transposons present in genomic DNA of the untransformed bacterial strain. Instead, IS1 insertion into this predetermined IS1 insertion region occurs after transformation of the plasmid DNA into the bacterial cell. RFLP profiles, see infra Example 2, reveal a correlation between low-producing DNA vaccine clones and an increased number of IS1 copies within the bacterial genomic DNA. Thus, the present invention further relates to methods for selecting a highly productive clonal subtype of a strain of E. coli harboring a plasmid DNA comprising detecting the presence or absence of one or more IS1 transposon insertion sequences within a region of the genomic DNA of said clonal subtype predetermined as a region of IS1 insertion (i.e., a “predetermined IS1 insertion region”), wherein a clonal subtype lacking IS1 transposon sequences within said IS1 insertion region represents a potential highly productive clonal subtype.

To this end, the present invention relates to a method for selecting a highly productive clonal subtype of a strain of E. coli harboring a plasmid DNA comprising: (a) detecting the presence or absence of an IS1 transposon sequence within a predetermined IS1 insertion region of the genomic DNA of said clonal subtype, wherein a clonal subtype lacking an IS1 transposon sequence within said insertion region represents a potential highly productive clonal subtype; and, (b) testing productivity of said potential highly productive clonal subtype; wherein a highly productive clonal subtype exhibits a high plasmid copy number per cell. A PCR-based assay, including but not limited to a quantitative PCR assay (“Q-PCR”), can be used to detect the presence or absence of an IS1 transposon sequence within said predetermined IS1 insertion region of the genomic DNA. A process for determining the location of an IS1 insertion region within genomic DNA of bacterial clonal subtypes is described in detail in Example 5, infra.

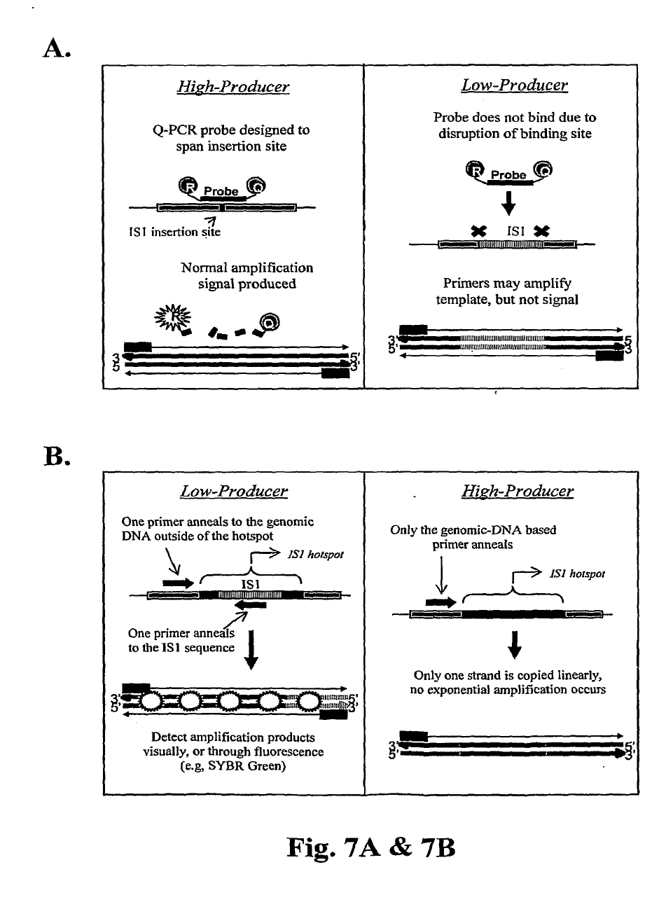

In one embodiment of the present invention, a Q-PCR assay is used to detect an increase of IS1 insertional mutagenesis within a portion of the genomic DNA of an E. coli clonal subtype predetermined to accept IS1 insertions after transformation of said E. coli, wherein said predetermined IS1 insertion region spans less than about 20 contiguous nucleotides of said genomic DNA. If said IS1 insertion regions spans less than about 20 contiguous nucleotides of said genomic DNA, said region will be referred to herein as a specific “IS1 insertion site.” A TaqMan Q-PCR assay is contemplated that detects the presence or absence of IS1 insertion sequences within this IS1 insertion site by attempting to amplify a portion of the genomic DNA that contains this predetermined IS1 insertion region (i.e., the IS1 insertion site); see a schematic diagram of the contemplated assay in FIG. 7A. Normal amplification and signal production only occurs when no IS1 sequences have inserted into the predetermined IS1 insertion site when using a fluorescent probe designed to span the IS1 insertion site.

Thus, in one embodiment of the present invention, a Q-PCR assay is contemplated which comprises amplification of a region of the genomic DNA predetermined to accept IS1 transposon sequences in the presence of a nucleic acid polymerase and a set of oligonucleotides consisting of: (i) a fluorescent probe labeled with a quencher molecule and a fluorophore which emits energy at a unique emission maxima, wherein said probe hybridizes to a location within the genomic DNA that spans the IS1 insertion region only when said genomic DNA lacks an IS1 transposon sequence within said IS1 insertion region; (ii) a forward PCR primer that hybridizes to a location of the genomic DNA upstream of the fluorescent probe; and, (iii) a reverse PCR primer that hybridizes to a location of the genomic DNA downstream of the fluorescent probe. When the fluorescent probe hybridizes to the genomic DNA, the nucleic acid polymerase will digest the probe during amplification to dissociate said fluorophore from said quencher molecule, and a change of fluorescence upon dissociation of the fluorophore and the quencher molecule is detected. This change of fluorescence corresponds to the amplification of the genomic DNA and, in turn, confirmation that the IS1 insertion site does not contain an IS1 transposon sequence. Thus, a change in fluorescence indicates the identification of a potential highly productive clonal subtype whose specific productivity can be subsequently evaluated in a small-scale fermentation system to confirm whether it is indeed a high-producing clone. Alternatively, hybridization of only the forward and reverse PCR primers to the genomic DNA of putative low-producing clones (i.e., clones that contain an IS1 transposon within the predetermined IS1 insertion site) will likely amplify the genomic template, but since the fluorescent probe can not hybridize to the IS1 insertion site, no fluorescent signal will be detected (see FIG. 7A). Importantly, this assay does not require multiplexing and can be performed with a whole cell lysate, eliminating the need for isolating genomic DNA. Since TaqMan probes typically vary in length from about 15 to about 40 nucleotides, to use this assay, the identified IS1 insertion site must be localized to a relatively narrow region of the genomic DNA sequence, preferably within less than about 20 contiguous nucleotides, to ensure adequate binding of the genome-specific probe.

In a further embodiment of the present invention, a PCR-based assay is used to detect an increase of IS1 insertional mutagenesis within a portion of the genomic DNA of an E. coli clonal subtype predetermined to accept IS1 insertions after transformation, wherein said predetermined IS1 insertion region spans greater than about 20 contiguous nucleotides of said genomic DNA; see a schematic diagram of the contemplated assay in FIG. 7B. If said IS1 insertion region spans greater than about 20 contiguous nucleotides of said genomic DNA, said region will be referred to herein as an “IS1 insertion hotspot.” The contemplated PCR assay utilizes one PCR primer that will hybridize to a location of the genomic DNA outside of the IS1 insertion region (i.e., outside of the IS1 insertion hotspot) and a second PCR primer that will hybridize to an IS1 transposon sequence of the genomic DNA located within the IS1 insertion hotspot. Hybridization of both primers will generate exponential amplification of fragments of the primer template of an approximate known target length and, in turn, result in the identification of a putative low-producing clone. Alternatively, in the absence of an IS1 transposon sequence within the IS1 insertion hotspot, only one primer will hybridize to the genomic DNA, resulting in the linear amplification of one strand of the template DNA and, in turn, the identification of a potential highly-productive clonal subtype. While it is possible that the second PCR primer, ideally intended to hybridize to an IS1 transposon sequence within the IS1 insertion hotspot, may hybridize to an IS1 transposon sequence outside of said insertion hotspot (i.e., a baseline IS1 transposon present within the genome of untransformed cells), by knowing the specific locations within the genomic DNA to which the two PCR primers will hybridize, as well as the location of the IS1 hotspot, the target length of amplified DNA from a putative low-producing clone can be easily approximated when said assay is performed using a whole cell lysate or purified genomic DNA.

Thus, in one embodiment of the present invention, a PCR assay is contemplated that will detect the presence or absence of IS1 transposon sequences within a predetermined IS1 insertion region using a set of oligonucleotides consisting of: (i) a first PCR primer that hybridizes to a location of the genomic DNA outside of the IS1 insertion region (i.e., outside of the IS1 insertion hotspot); and, (ii) a second PCR primer that hybridizes to a location within an IS1 transposon sequence in the predetermined IS1 insertion region; wherein the presence of an IS1 transposon sequence within the IS1 insertion region results in exponential amplification of said genomic DNA due to the hybridization of and amplification from both PCR primers, and the absence of an IS1 transposon sequence within the IS1 insertion region results in linear amplification of one strand of genomic DNA due to hybridization of and amplification from only the first PCR primer. The exponential amplification of the genomic DNA can be visually detected by identifying amplified nucleic acid fragments of approximate target size or fluorescently detected in real-time by adding a nucleic acid stain that binds to double-stranded amplified DNA (e.g., SYBR® Green). Alternatively, a fluorogenic primer (e.g., the LUX™ primer (Invitrogen)) can be used to measure exponential increases in fluorescence, keeping in mind that a fluorescent signal will be expected even in clones lacking a IS1 transposon sequence within the IS1 insertion hotspot; however, the signal will increase linearly in such a situation, instead of exponentially as would result from hybridization of both PCR primers. The assay must also account for the possibility of insertion of IS1 transposon sequences within the IS1 insertion hotspot in either orientation. Thus, to account for this possibility, internal IS1 primers in both directions can be used, running either two separate assays per sample with the individual primers or using both primers to screen the population of clones.

Because the low-producer phenomenon is correlated with increased IS1 insertional mutation, an E. coli host strain devoid of all IS1 copies will likely result in a more uniform population of highly-productive clones. To this end, the present invention further relates to a method of generating a highly productive clonal subtype of a strain of E. coli harboring a plasmid DNA comprising mutating an E. coli host strain to remove all copies of IS1 from the bacterial genome prior to transformation of the bacterial strain with said plasmid DNA. The present invention further relates to both a mutated E. coli host strain, including but not limited to a mutated DH5 strain, wherein all IS1 copies have been removed, and the use of said strain for the propagation of plasmid DNA.

Several methods exist for the construction of deletion or disruption mutations of E. coli including P1 phage transduction, transposon-mediated random mutagenesis, and generalized (RecA-mediated) homologous recombination. These methods are typically only suitable for single mutations due to the need for a selectable marker, e.g. antibiotic resistance, for each mutation. An alternative method involves the use of PCR products with 36- to 50-nt extensions on the primers that are homologous to the flanking sequences around the desired disruption site, and the lambda-Red recombinase (Datsenko and Warmer, 2000, PNAS 97:6640-6645). A selectable marker is still used in the case; however, the marker can be subsequently removed, freeing its use for additional rounds of mutation. A modified method that eliminates residual “scars” utilizes the endogenous double-strand break repair process to remove the selectable marker (Kolisnychenko et al., 2002, Genome Res. 12:640-647). This method was used to produce a K-12 strain of E. coli with an 8.1% reduction in genome size, including elimination of 24 of 44 transposable elements. Three of the seven IS1 copies were removed in this strain. It is highly probable that removal of the remaining 4 copies will have no deleterious effects on the survivability of the strain or suitability of its use in the fed-batch fermentation process. Note, however, that the modified method of Kolisnychenko et al. is not suitable for E. coli strain DH5 due to the need for RecA in the double-strand break repair process. Another method utilizes group II introns, so-called “targetrons,” to produce mutations based on 14- to 16-nt regions of complementary sequence (Zhong et al., 2003, Nucleic Acids Res. 31:1656-1664). This method also utilizes a selectable marker than can be subsequently removed to allow for multiple insertions. However, it does not produce deletions of the target sites as the two previous methods, but rather produces disruptions. Use of this method would result in a strain that carries 7 non-functional copies of IS1, being disrupted in the main transposase gene (insAB) encoded by the transposon.

The plasmid DNA vector contained within the transformed E. coli clones described herein can be any extra-chromosomal DNA molecule containing a gene(s) encoding a biological compound of interest, i.e. a transgene(s). The plasmid will contain elements required both for its maintenance and propagation in a microbial cell (e.g., E. coli), as well as for the subsequent expression of the transgene in the animal host. For bacterial propagation, an origin of replication is needed, in addition to any plasmid encoded function required for replication, such as a selectable marker for selection of successful transformants. For gene expression, the plasmid should be designed to maximize transient production of the transgene upon entry into the animal host. Components of the plasmid contributing to gene expression may include, but is not limited to, a eukaryotic promoter, a transcriptional termination and polyadenylation signal, and an enhancer element(s). A selected promoter for recombinant gene expression in animal cells may be homologous or heterologous, and may be constitutive or inducible, including but not limited to promoters from human cytomegalovirus/immediate-early (CMVIE), simian virus/early (SV40), human elongation factor-1α (EF-1α) and human ubiquitin C (UbC). Plasmid DNA can be recombinantly engineered using techniques well known to those of ordinary skill in the art. See, e.g., Sambrook, et al., supra; and Current Protocols in Molecular Biology, Greene Publishing Assoc. & Wiley (1987); both of which are incorporated by reference herein.

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying figures, it is to be understood that the invention is not limited to those precise embodiments, and that various changes and modifications may be effected therein by one skilled in the art without departing from the scope or spirit of the invention as defined in the appended claims.

The following examples are provided to illustrate the present invention without, however, limiting the same hereto.

### EXAMPLE 1

### Identification of IS1 Transposon in DNA Vaccine Plasmid

Strains, DNA vaccine plasmids and growth media—The host strain for all DNA vaccine constructs is E. coli DH5 [F⁻ deoR recA1 endA1 hsdR17(rₖ⁻, mₖ⁺) supE44λ⁻ thi-1 gvrA96 relA1]. The strain was originally purchased from Invitrogen (Carlsbad, Calif.; formerly Gibco BRL), adapted in the defined medium DME-P5, and made electrocompetent for subsequent transformations. E. coli DH5α [F⁻ φ80lacZΔM15 Δ(lacZYA-argF) U169 deoR recA1 endA1 hsdR17(rₖ⁻, mₖ⁺) gal⁻ phoA supE44λ⁻ thi-1 gyrA96 relA1] was purchased as electro-competent cells from Invitrogen (Carlsbad, Calif.). Construction of the HIV DNA vaccine plasmid V1Jns-nef is described in detail in International PCT Application Number PCT/US00/34162, filed Dec. 15, 2000 (published as International Publication Number WO 01/43693 on Jun. 21, 2001). Briefly, the DNA vaccine plasmid consists of a pUC19-derived bacterial origin of replication and neomycin/kanamycin resistance gene (nptII) for maintenance and selection in E. coli; and a CMV-IE promoter, intron A and bovine growth hormone terminator/polyadenylation signal for eukaryotic expression of the HIV-derived transgenes. Transformations were performed by electroporation according to standard practices. Dehydrated LB broth and LB agar were purchased from Becton-Dickinson (Franklin Lakes, N.J.) and prepared according to manufacturer's instructions. Sterile SOC medium (for post-transformation recovery) was purchased from Invitrogen. Defined medium DME-P5 contains the following: 7 g/l KH₂PO₄, 7 g/l K₂HPO₄, 6 g/l (NH₄)₂SO₄, 5 g/l L-Glutamic Acid, 10 g/l glycerol, and 0.5 g/l NaCl, adjusted to pH 7.2 with NaOH. 8.3 ml Neomycin/Thiamine/MgSO₄ solution and 1 ml trace elements solution were added per liter post-sterilization. The 120× Neomycin/Thiamine/MgSO₄ solution contains 24 g/L Thiamine-HCl, 240 g/l MgSO₄.7H₂O, and 9.6 g/l Neomycin Sulfate. The 1000× trace elements solution was dissolved in 1.2N HCl and contains 27 g/l FeCl₃.6H₂O, 2 g/l ZnCl₂, 2 g/l CoCl₂.6H₂O, 2 g/l Na₂MoO₄.2H₂O, 1 g/l CaCl₂.2H₂O, 1.3 g/l CuCl₂.2H₂O, and 0.5 g/l H₃BO₃.

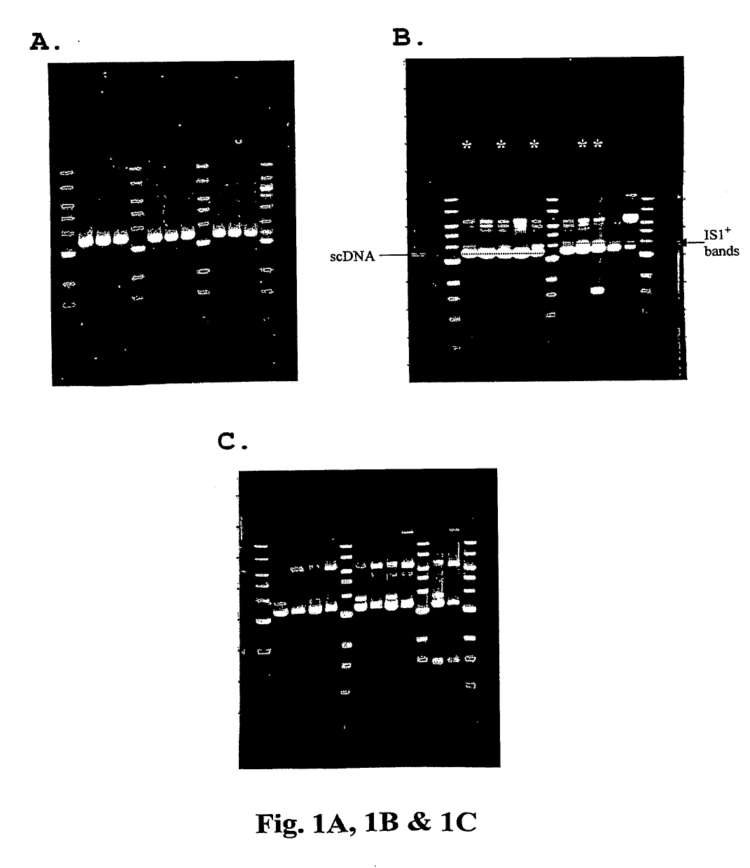

Results—E. coli DH5 prepared in LB medium was transformed with 1 μl of V1Jns-nef and recovered in SOC medium. Transformants were plated on LB/neomycin agar. Ten colonies (NLB-1 to NLB-10) chosen at random were used to inoculate 10 ml LB/neo liquid medium and grown overnight. One milliliter aliquots were removed for isolation of plasmid DNA, and frozen glycerol stocks were also prepared. Fresh LB cultures were prepared using the glycerol stocks as inocula, and growing LB cultures were used to inoculate 25 ml DME-P5 medium in shake flasks. The cultures were subjected to three rounds of passaging in DME-P5 for adaptation to the medium shift. After the third round, 1 ml aliquots were withdrawn and the plasmid DNA was isolated with the QIAGEN plasmid mini-prep kit. DNA samples from the LB-grown and DME-P5-grown cultures were run on 0.7% agarose gels to verify plasmid content (FIGS. 1A and 1B, respectively). Comparison of the two gels reveals that several of the DME-P5 samples contain a minor, higher molecular weight species, relative to the dominant species, that is not evident in the LB samples (see NLB-1, -3, -5, -7 and -8; starred lanes in FIG. 1B). One sample, NLB-8, also contains a major, lower molecular weight species that is most likely formed from a rearrangement and exclusion of DNA from the original molecule. Sample NLB-10 is unique in that it appears to exist predominantly as a dimerized molecule, representing either a covalent linkage or merely a strong physical association.

The presence of two species of differing molecular weights was observed previously in a similar culture of a DNA vaccine plasmid comprising HIV gag that failed to amplify plasmid DNA following fed-batch cultivation with an extended slow-growth phase (labeled a “low-producer” or “LP”) (data not shown). Subsequent to that observation, the transposable element IS1 was discovered in samples of a similar DNA vaccine plasmid. To examine the possibility of IS1 insertion in the DME-P5-grown culture preps of V1Jns-nef, the five NLB DNA samples containing the minor, higher molecular weight species were subjected to restriction enzyme digestion with MluI. Restriction digests were set up as follows: 1 μl plasmid DNA, 7 μl H₂O, 1 μl 10× reaction buffer, 1 μl enzyme. Digestions were incubated at 37° C. for ˜1 hour, and then 2 μl of loading buffer was added to each for loading onto the gel. Based on a comparison of the nucleotide sequences, this enzyme should cut within the IS1 fragment but not within the V1Jns-nef sequence. Hence, if the higher molecular weight bands contain IS1, MluI restriction enzyme digestion should result in the linearization and migration of these bands on an agarose gel. Non-IS1-containing species should not migrate relative to the undigested samples. Indeed, after MluI digestion, all five of the samples examined displayed the characteristic migration of the higher molecular weight bands consistent with linearization, while the lower bands did not shift (FIG. 1C).

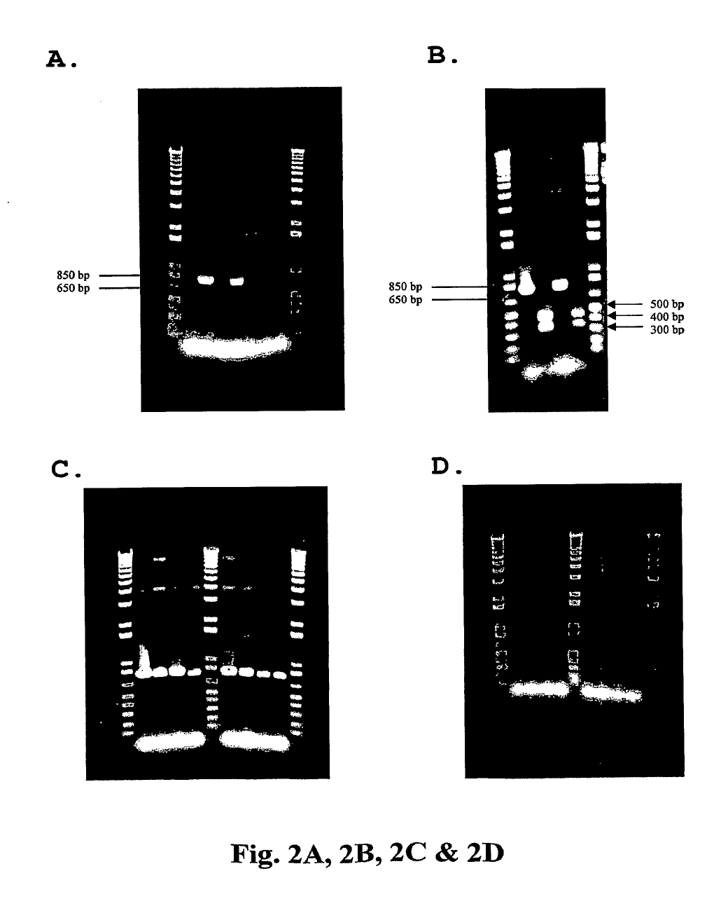

Oligonucleotide primers were designed for amplification of the IS1-containing fragment to provide additional evidence of its presence. The designed primers were so-called “imperfect match” primers consisting of 9 bp of non-complementary nucleotides followed by 7 or 9 bp of nucleotides complementary to the ends of the IS1 sequence. Primers designed in this manner had the net effect of being sensitive to template concentration. Therefore, only samples with an IS1 content greater than an unspecified amount would be amplified. Samples NLB-1 and NLB-2 from LB- and DME-P5-grown cells were chosen as templates for PCR reactions. The higher molecular weight band was not evident on agarose gels in either NLB-1 or NLB-2 isolated from LB-grown cells; however, it was evident in NLB-1, but not in NLB-2, from DME-P5-grown cells. There was no evidence of higher MW bands in pUC19 samples (data not shown); hence, this was included as a negative control. Presumably, IS1 transposes from the genome into the plasmids; therefore, genomic DNA preps were conducted for E. coli DH5 and DH5α. Both strains were grown in LB prior to isolation of genomic DNA. PCR reactions were established in 50 μl total volume using the HotStarTaq™ Master Mix reagent from QIAGEN as per the manufacturer's instructions. 5 μl of each reaction was run on a 0.7% agarose gel (FIG. 2A). Consistent with the agarose gel analysis, there was no significant amplification of a fragment corresponding in length to IS1 from either of the LB-grown preps of NLB samples (lanes 2 and 4). However, a fragment migrating between 650 and 850 bp was amplified from both the NLB-1 and NLB-2 preps of DME-P5-grown cultures (lanes 3 and 5). There was no amplification from pUC19 or either of the genomic DNA preps, confirming the inability of the primers to amplify IS1 found in contaminating residual genomic DNA in these preparations of plasmid DNA tested. To confirm the identity of the amplified fragments, the two positive samples were subjected to MluI restriction enzyme digestion (3 μl PCR reaction, 5 μl H₂O, 1 μl 10× reaction buffer, 1 μl enzyme, incubated 2 hours at 37° C.). This enzyme should cut IS1 once, producing two bands of ˜0.44 and ˜0.34 Kb, and indeed, this was observed for the amplified samples (FIG. 2B).

The presence of IS1 in the DME-P5-grown prep of the NLB-2 sample raised the possibility that the insertion sequence could be present in a significant, though still very minor, percentage of the population in all of the DME-P5-grown cells. To test this possibility, PCR reactions were conducted using the NLB-3 through NLB-10 samples from both LB- and DME-P5-grown cells as templates. The results indicate that the IS1 fragment is indeed present in all ten of the DME-P5-grown samples of the NLB preps (FIG. 2C). The results from the LB-grown preps are less clear (FIG. 2D). There are faint bands between 650 and 850 bp in all of these samples; however, the degree of amplification suggests that the IS1 is present in extremely small amounts, and may even result from chromosomal contamination of the plasmid prep. These faint bands may also represent extension resulting from non-specific binding of the primers. However, it is clear that the IS1 sequence is present in DME-P5 preps. It is also highly likely that the shift from LB to DME-P5 medium induces an increase in IS1 among the plasmid population.

The NLB samples passaged in DME-P5 medium were then cultured in the SFF (see International Application No. PCT/US20051002911; supra) assembly to examine plasmid DNA content. All 10 of the samples were characterized as “low-producers,” with plasmid DNA content ranging from 0.8 to 2.4 μg DNA/OD₂ pellet. The identification of the IS1 transposable element in each of these samples raises the question of whether the presence of IS1 is responsible for the low-producer phenomenon.

### EXAMPLE 2

### Comparison of IS1 Content in High- and Low-Producer Genomes Using Restriction Fragment Length Polymorphism (RFLP) Analysis

Strains, DNA vaccine plasmids and growth media—See, supra, Example 1. Additionally, unadapted, untransformed cells were purchased from Invitrogen and maintained in LB medium. Construction of the HIV DNA vaccine plasmid V1Jns-tpa-nef (5540 bp) is described in detail in International PCT Application Number PCT/US00/34162 (supra). Construction of the HIV DNA vaccine plasmid V1Jns-tpa-pol (7516 bp) is described in detail in International PCT Application Number PCT/US00/34724, filed Dec. 21, 2000 (published as International Publication Number WO 01/45748 on Jun. 28, 2001). Construction of the HIV DNA vaccine plasmid V1Jns-tpa-gag (6375 bp) is described in detail in International PCT Application Number PCT/US98/02293, filed Feb. 3, 1998 (published as International Publication Number WO 98/34640 on Aug. 13, 1998).

Shake flask cultivation of and DNA isolation from HIV DNA vaccine cultures—The HIV DNA vaccine high-producer (-HP) and the low-producer (-LP) clones of V1Jns-tpa-gag were isolated previously using the standard High-Producer Screen as described in International Application No. PCT/US2005/002911 (supra). 25 μl aliquots of frozen glycerol stocks were used to inoculate 25 ml DME-P5 in 250-ml shake flasks. Cells were grown overnight at 37° C., 220 RPM in a Kuehner cabinet shaker and harvested for isolation of DNA in mid- to late-exponential phase. Low-producer (-LP) clones of V1Jns-tpa-pol and V1Jns-tpa-nef were prepared by first transforming adapted E. coli DH5 cells with purified plasmid DNA. Transformants were recovered in and plated on DME-P5, and 5-10 single colonies were selected for growth in shake flasks. The cells were harvested in mid- to late-exponential phase and used to inoculate a new round. Cultures were passaged in this manner for a total of three rounds. The failure to amplify plasmid DNA in the candidate low-producers was confirmed by fed-batch cultivation in shake flasks, as described in International Application No. PCT/US2005/002911 (supra), and a single clone for each construct was stored as a representative low-producer. 25 μl aliquots of these frozen glycerol stocks were used to inoculate flasks and harvest cultures for DNA isolation as described above. Total DNA was isolated using the Promega Wizard® Genomic DNA Purification Kit (Madison, Wis.). DNA pellets were rehydrated in 10 mM Tris.HCl, pH 8.5. Plasmid DNA from each high- and low-producer sample was isolated using the Qiagen Miniprep Spin Kit (Valencia, Calif.).

Restriction Fragment Length Polymorphism (RFLP) analysis—An IS1-specific probe (0.7 Kb) was created using the PCR DIG Probe Synthesis Kit (Roche Molecular Biosystems, Mannheim, Germany) to incorporate the non-radioactive, modified nucleotide DIG-11-dUTP into the DNA by PCR. Primers were ordered from Sigma Genosys (The Woodlands, Tex.) and plasmid plS1 was used as template DNA. Primer sequences were as follows: IS1—F2,5′-GGTAATGACTCCAACTTATTG-3′ (SEQ ID NO:1); IS1-R2, 5′-GGTGATGCTGCCAACTTA-3′ (SEQ ID NO:2). The PCR conditions used were as per manufacturer's suggestions. Restriction enzymes for digestion of DNA were purchased from New England Biolabs (Beverly, Mass.). Digested total and plasmid-only DNAs from each sample were run on 0.7% agarose gels overnight (˜16 hours) at 34 V, 4° C. DNA was transferred onto Nytran SuPerCharge nylon membranes for 1 hour using the Schleicher and Schull Turboblotter™ Rapid Downward Transfer System (Keene, N.H.) as per manufacturer's protocol. DNA was crosslinked to membranes by UV irradiation at 150 mJoule using the BioRad GS Gene Linker® (Hercules, Calif.). The DIG-labeled IS1 probe was hybridized to the target DNA on Southern blots following the Filter Hybridization Protocol with overnight incubation (Roche Molecular Biosystems, Mannheim, Germany). Probe-target hybrids were visualized by an enzyme-linked chemiluminescent assay using an anti-DIG alkaline phosphatase antibody and CSPD, an alkaline phosphatase substrate (Filter Hybridization Protocol, Roche Molecular Biosystems, Mannheim, Germany).

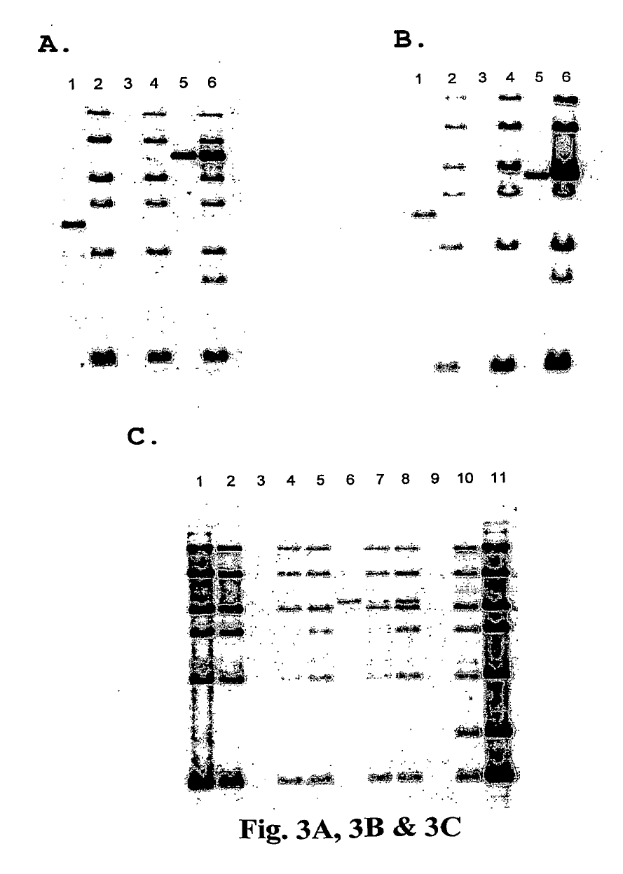

Results—IS1 RFLP profiles of V1Jns-tpa-pol clones—The enzyme pair AflI and AgeI was used to generate fragments for IS1-specific RFLP analysis of high- and low-producer clones of the DNA vaccine construct V1Jns-tpa-pol. The high-producer clone (tpa-pol-HP) was previously isolated through the standard screening process described in detailed in International Application No. PCT/US2005/002911 (supra) and was obtained as a frozen working seed vial. Total DNA preps from high-producer (tpa-pol-HP) and low-producer (tpa-pol-LP) cultures grown in DME-P5 were digested with restriction enzymes AflII and AgeI, while plasmid DNA isolated from each culture was digested with AflII. As a control, genomic DNA was also isolated from DH5 cells grown in LB medium with no prior adaptation to DME-P5. A Southern blot was prepared and hybridized with the DIG-labeled IS1 probe (FIG. 3A). Six IS1-containing fragments appear in both the DH5 (lane 2) and tpa-pol-HP (lane 4) samples. The lowest molecular weight band is approximately two-fold more intense than the other bands, suggesting that this band may contain more than one IS1 insertion sequence. However, this fragment is less than 2 Kb and may not be large enough to accommodate two 768-bp IS1 insertions. The higher intensity of the lowest molecular weight band could indicate overlapping IS1 containing fragments or reflect a better transfer efficiency of smaller fragments onto the Nytran membrane relative to higher molecular weight bands. Therefore, the IS1 copy number of untransformed DH5 could be as low as between 6 and 7. The IS1 profile of tpa-pol-LP (lane 6) contains two additional IS1-positive bands not found in either the DH5 control or tpa-pol-HP samples. Based on a comparison with the tpa-pol-LP plasmid only control (lane 5), the higher molecular weight band of these two (7-8 kb) is consistent with plasmid DNA. However, the lower, 34 kb band is not found in any other samples and is therefore a strong indication that an additional site of IS1 insertion is present in the tpa-pol-LP genome.

IS1 RFLP profiles of V1Jns-tpa-nef clones—V1Jns-tpa-nef clones were profiled for IS1 insertions using the enzymes AflI and AgeI (FIG. 3B), with results that are similar to those obtained with the V1Jns-tpa-pol clones. Six IS1-containing bands are evident in both the DH5 control (lane 2) and the tpa-nef-HP (lane 4) samples, with the smallest band present at a higher intensity than the others. In the tpa-nef-LP sample, 7 bands are visible, with the third largest band clearly more intense than the others (lane 6). Comparison to the plasmid only sample for this clone (lane 5) shows that the IS1-containing plasmid runs very close to a genomic DNA fragment carrying IS1. Therefore, the highest intensity band is actually the result of overlapping bands of plasmid and genomic DNA fragments. There remains, however, a 3-4 kb band that is not found in either the DH5 control or the tpa-nef-HP sample and is approximately the same size as the unidentified band observed in the tpa-pol-LP sample above. This is another indication of an IS1 insertional mutation in low-producer genomes.

IS1 RFLP profiles of V1Jns-tpa-gag clones—Since AflII-AgeI IS1 profiles of both V1Jns-tpa-pol and V1Jns-tpa-nef low-producer clones indicated an additional IS1 insertion site within the genomes of the low-producers, V1Jns-tpa-gag clones were also examined to determine whether mutations were present in all three V1Jns-tpa constructs. In this case, however, the source of the high- and low-producer clones differed from that of V1Jns-tpa-pol and V1Jns-tpa-nef. Both clones were isolated many years ago and have been propagated as laboratory seed since that time to serve as controls in the High-Producer Screen at the shake flask fermentation with feeding (SFF) stage (lab seed sample) (see International Application No. PCT/US2005/002911; supra). Thus, the exposure time of these clones to DME-P5 is much higher than that for the tpa-pol and tpa-nef clones. A frozen working seed vial comparable in generation time to the previously evaluated high-producers was also obtained for analysis (working seed sample). The RFLP results show that the IS1 profiles of total DNA from both high-producer clones are similar (FIG. 3C), although there is a faint band in the lab seed sample (lanes 7 and 8) that runs slightly higher than the third largest fragment in the working seed sample (lanes 4 and 5). This band is also present in the plasmid only sample of the high-producer lab seed (lane 6), so it can be attributed to plasmid DNA. A much lighter band is barely visible in the plasmid only sample of the working seed (lane 3). This RFLP analysis is consistent with Q-PCR results (see, infra, Example 4), indicating that the fraction of IS1-containing plasmids may increase with increasing cultivation time of high-producers in defined medium. The genome profiles of both high-producer samples are also similar to the unadapted DH5 control (lane 1).

A tpa-gag low-producer equivalent to the tpa-pol and tpa-nef samples described previously was not available, so only the lab seed was evaluated in this case (FIG. 3C). The RFLP results show that, as with tpa-pol-LP and tpa-nef-LP, an additional band is present in the tpa-gag-LP IS1 profile that does not correspond to plasmid DNA (lanes 10 and 11). However, this band is 2-3 kb, smaller than that observed in the other clones. Unlike tpa-pol and tpa-nef, the IS1 probe did not bind to plasmid DNA isolated from the tpa-gag-LP sample (lane 9). In its role as the low-producer control for the SFF stage of the High-Producer Screen, the tpa-gag-LP lab seed consistently yields <<I mcg plasmid DNA/mg dry cell weight (DCW), whereas the tpa-pol and tpa-nef low-producers contained ˜2 mcg plasmid DNA/mg DCW. The latter value is more typical of a low-producer selected at random. Therefore, it is possible that while the insertional mutation found in tpa-gag-LP is distinct from those found in tpa-pol-LP and tpa-nef-LP, it results in a copy-number suppressed phenotype that has an even greater impact on the ability of a clone to amplify plasmid DNA.

IS1 RFLP profile of E. coli DH5 adapted to DME-P5—The IS1 profiles of high- and low-producers of three different DNA vaccine constructs indicate that all the low-producer clones contain an IS1 insertional mutation whereas high-producers are similar to unadapted DH5. It is possible that the adaptation results in an increase in the copy number of IS1 in the genome. To test this, IS1 profiles of both unadapted DH5 and DH5 adapted to DME-P5 were analyzed following digestion of the genomic DNA with AflII and AgeI (FIG. 3C). The RFLP results indicate that there are no differences between the locations of IS1 in the two E. coli genomes (lanes 1 and 2). Therefore, it appears that low-producers are correlated with IS1 insertional mutation of the genome, and that this insertion occurs following the transformation step.

### EXAMPLE 3

### IS1 Transposon Integration Sites in V1Jns Plasmids

Material and Methods—Plasmid DNA from V1Jns-nef clone NLB-5 propagated in DME-P5 medium was obtained as described in Example 1. A total of sixteen oligonucleotide primers complementary to the full, insertion-free plasmid were designed to anneal in ˜700-bp increments in both the forward (8) and reverse (8) directions. A second set of primers were specific to the forward and reverse ends of the IS1 insertion sequence. A series of 32 PCR reactions were established consisting of (i) one of the 16 V1Jns-nef-specific primers and one of the 2 IS1-specific primers, (ii) clone NLB-5 plasmid DNA as template, (iii) and HotStarTaq PCR Master Mix Reagent (Qiagen). The PCR reactions were run using standard protocols. Each sample was analyzed on a 0.7% agarose gel to identify amplified fragments. The presence of an amplified fragment is a preliminary indication of a vector-IS1 junction, but does not eliminate the possibility of mis-priming events (false positives). Since the primers utilized covered both strands of the plasmid DNA and both possible orientations of insertion of transposons, the presence of a second amplified fragment consistent with the amplification of the same insertion on the complementary strand was used to reduce the likelihood of false positives. The sizes of the confirmed amplified fragments provided a preliminary insertion map. Several amplified fragments were then selected for cloning into the pCR®2.1-TOPO® vector (Invitrogen), and were subsequently sequenced using an Applied Biosystems 310 Genetic Analyzer to identify the precise location and orientation of IS1 insertions.

Results—Using a series of PCR reactions with one IS1-specific primer and one V1Jns-specific primer followed by sequencing of the amplified products, the locations of S1 insertions were identified using the NLB-5 clone (see Example 1). Both partial and full junctions were resolved, and sequencing confirmed that the transposon produces a 9-bp target duplication at the site of insertion. Eleven unique integration sites were identified, with insertion of the transposon in both orientations; one site of insertion was observed in both orientations. All sites were in or within 85 base-pairs of the coding region of the neomycin resistance gene, nptII. Since IS1 insertions were not found in pUC-neo clones constructed by replacing the amp^{R} gene (bla) of pUC19 (New England Biolabs) with a neo^{R} gene (nptII) from pUC4K (Amersham Pharmacia; Piscataway, N.J.), the presence of the neomycin resistance gene alone is not sufficient to induce transposition into the plasmid molecule.

### EXAMPLE 4

### Relative Quantitation of IS1 Based on Plasmid Copy Number Using Real-Time Q-PCR

Strains, DNA Vaccine Plasmids and Growth Media—See, supra, Examples 1 and 2.

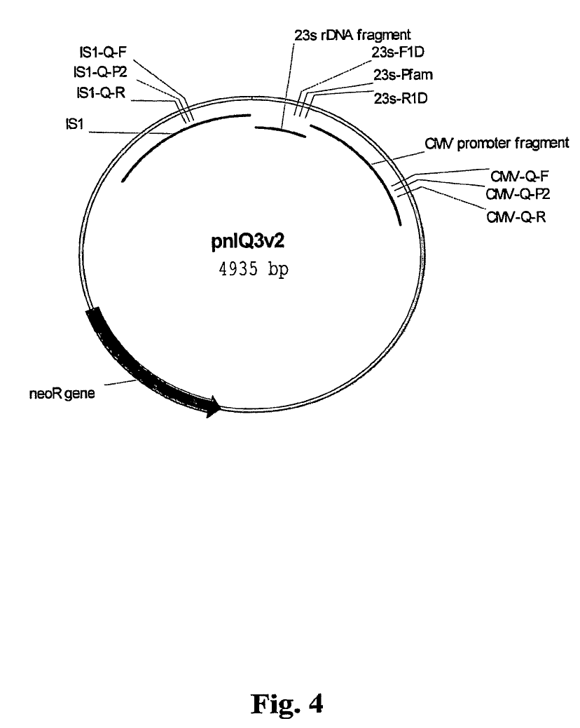

Construction of plasmid standards—All molecular biology manipulations were performed according to standard protocols (Sambrook et al., 1989, supra). Enzymes were purchased from New England Biolabs (Beverly, Mass.) pUC-neo was constructed by replacing the amp^{R} gene (bla) of pUC19 (New England Biolabs) with a neo^{R} gene (nptII) from pUC4K (Amersham Pharmacia; Piscataway, N.J.). The 768-bp sequence of IS1 was PCR-amplified from a sample of plasmid V1Jns-nef containing the transposon. The fragment was cloned into the pCR®2.1-TOPO® vector (Invitrogen) to create IS1 plasmid standard pIS1, then excised using restriction enzyme EcoRI, and ligated into the EcoRI restriction site of pUC-neo using T4 DNA ligase to obtain plasmid standard pnIQ1. A partial CMV promoter was extracted as a 0.7 Kb SpeI-SphI fragment from V1Jns-nef and ligated to pnIQ1 double-digested with XbaI and SphI to obtain plasmid standard pnIQ2. A fragment of 23s rDNA was PCR-amplified from DH5 genomic DNA using primers designed for the E. coli K-12 sequence in GenBank (Accession Number M25458) as follows: 23s-F1 (5′-GGATCCAACCCAGTGTGTTTCGACAC-3′; SEQ ID NO:9) and 23s-R1 (5′-GGATCCAGACAGGATACCACGTGTCC-3′; SEQ ID NO: 10). BamHI restriction sites (underlined) were included on either end of the 23s rDNA fragment to facilitate ligation. The 0.3 Kb PCR fragment was cloned into the pCR®2.1-TOPO® vector (plasmid p23sTA), then excised with BamHI and ligated into the BamHI site of pnIQ2 to obtain the final plasmid standard pnIQ3v2 (FIG. 4). This plasmid contains the full IS1 sequence and portions of the CMV promoter and 23s rDNA sequences, all of which are targets for the Q-PCR assays. DNA concentrations of plasmid standards were determined by UV absorbance at 260 nm (A₂₆₀=1≅50 μg/mL), and dilutions were prepared to obtain standard solutions with 10³-10⁸ copies/μL.

Shake flask cultivation of DH5 (V1Jns-nef)—See, supra, Example 2.

Real-time quantitative PCR—Sequence detection primers and probes were designed using Primer Express software v. 2.0 from Applied Biosystems (Foster City, Calif.). Unlabeled primers were purchased from Sigma-Genosys (The Woodlands, Tex.) and fluorescently-labeled probes were purchased from Applied Biosystems. TaqMan probes were designed to anneal between the primers on the template DNA and include reporter dyes 6-carboxyfluorescein (FAM) or the Applied Biosystems proprietary dye “VIC” at the 5′ end, and 6-carboxytetramethylrhodamine (TAMRA) at the 3′ end. Primers CMV-Q-R (5′-GTACGGTGGGAGGTCTATATAAGCA-3′; SEQ ID NO:3) and CMV-Q-R (5′-GGAGGTCAAAACAGCGTGGAT-3′; SEQ ID NO:4), and VIC-labeled TaqMan probe CMV-Q-P2 (5′-VIC-TCGTTTAGTGAACCGTCAGATCGCCTG-3′-TAMRA; SEQ ID NO:5), were designed to quantify plasmid DNA using the CMV promoter as a marker. Primers IS1-Q-F (5′-AGGCTCATAAGACGCCCCA-3′; SEQ ID NO:6) and IS1-Q-R (5′-ACGGTTGTTGCGCACGTAT-3′; SEQ ID NO:7), and FAM-labeled TaqMan probe IS1-Q-P2 (5′-FAM-CGTCGCCATAGTGCGTTCACCG-3′-TAMRA; SEQ ID NO:8), were designed to quantify IS1. The CMV and IS1 primer-probe sets were run in multiplex mode to quantitate total plasmid and transposon copies.

To develop a second assay for use in determining residual genomic IS1, primers and probes were designed to quantify 23s rDNA as follows: 23s-F1D (5′-GAAAGGCGCGCGATACAG; SEQ ID NO:11), 23s-R1D (5′-GTCCCGCCCTACTCATCGA; SEQ ID NO:12) and FAM-labeled TaqMan probe 23s-Pfam (5′-FAM-CCCCGTACACAAAAATGCACATGCTG-TAMRA; SEQ ID NO:13). For the residual genomic DNA assay, the 23s rDNA and CMV primer-probe sets (see above) were run in multiplex mode.

PCR was performed in 20 μL reaction volume with constant volumes of 10 μL of 2× Universal PCR Master Mix (Applied Biosystems) and 2 μL sample DNA, and various volumes of primers and probes. The 384-well plate format was utilized with six ten-fold dilutions of pnIQ3v2 standard, and four to six replicates per sample. Amplification and fluorescence detection of the samples was performed in an ABI 7900HT Sequence Detection System (Applied Biosystems) under the following thermal cycler conditions: 50° C. for 2 min, 95° C. for 10 min, followed by 40 cycles of 95° C. for 15 s and 60° C. for 1 min.

Analysis of real-time quantitative PCR—Data analysis was completed using the ABI Prism 7900HT Sequence Detection System (SDS) v. 2.0 software. The PCR threshold cycle number (C_{T}) was calculated from the point on the amplification plot where the fluorescence of the samples crossed a user-defined threshold limit. For absolute quantitation experiments, template copy number was calculated using standard curve plots of copy number vs. C_{T}. Relative quantitation of samples was performed using the 2^{-ΔΔC}^{_{T}} method (Livak and Schmittgen, 2001, Methods 25:402-408). Briefly, for the quantitation of sample q, normalized to an endogenous reference and relative to a calibrator sample cb,

$$\frac{{X}_{N , q}}{{X}_{N , cb}} = \frac{K \times {( 1 + E )}^{- Δ {C}_{T , q}}}{K \times {( 1 + E )}^{- Δ {C}_{T , cb}}} = {( 1 + E )}^{- Δ Δ {C}_{T}} = {2}^{- Δ Δ {C}_{T}} for E \approx 1 ,$$

where:

ΔC_{T}=C_{T,X}−C_{T,R}, the difference in threshold cycles for target and reference molecules,

ΔΔC_{T}=ΔC_{T,q}−ΔC_{T,cb},

X_{N}=normalized amount of target (Xₒ/Rₒ) relative to an endogenous reference (Rₒ), and

E=amplification efficiency of the reactions.

In these copy ratio assays, IS1 (or 23s rDNA) was the target sequence; the CMV promoter was the endogenous reference; and all unknown samples were compared to plasmid standards pnIQ2 or pnIQ3v2 as a calibrator (1:1 copy ratio of all targets). For this method to be accurate, two assumptions must be valid. First, the amplification efficiencies for all reactions must be approximately 100%. The design of primers and probes using the Primer Express software produced reagents with high efficiency to satisfy this requirement. Secondly, the efficiencies of both the target and endogenous reference must be near equivalent, with a difference below 0.1.

Sequence validation of CMV promoter region as a target for plasmid quantitation—In a previous scan of the complete V1Jns-nef plasmid, 11 unique sites of insertion for IS1 were identified, all of which were in or within 100 base-pairs of the coding region of the neomycin resistance gene and >550 base-pairs removed from the CMV promoter (see Example 3). To confirm the suitability of this promoter as a Q-PCR target (i.e., as a template free of IS1 insertion), this region of V1Jns-nef was retested for IS1 insertion. If insertion occurs within the intended amplification sequence, CMV primers and probes may not bind, and a significant error will arise within copy number ratio calculations. Using a sample of V1Jns-nef known to contain transposons as template DNA, PCR reactions were run with one of three primers complementary to the CMV promoter and one of two specific primers for the ends of IS1. Any resulting amplicons larger than the insertion sequence (768 bp) might contain IS1-plasmid junctions; however, non-specific binding of the primer may produce a false positive. By using primers at multiple points along the CMV promoter sequence, amplified fragments from non-specific binding can be eliminated if the complementary primer pairs do not produce analogous results. Five amplicons greater than 0.7 Kb were obtained and all but two were eliminated as a result of non-specific binding using the above criteria. The last two were sequenced, and the IS1 insertions were found to occur upstream of the CMV promoter at sites in or near the coding region of the neomycin resistance gene, with one of the two sites previously documented. Thus, the CMV promoter region can be used as a target for fluorescent probes during Q-PCR assays without concern for interference from transposons.

Primer-probe concentration optimization for single and multiplex reaction—Primer and probe concentrations for both sets of target sequences were optimized to achieve the lowest threshold cycle (C_{T}) with minimal amounts of reagents. Concentrations of IS1, 23s rDNA and CMV promoter probes were optimized to 200 nM for single and multiplex reactions in both assays. An optimal concentration of 100 nM for CMV-Q primers was found to reduce spectral interference during multiplexing in both IS1/CMV and 23s rDNA/CMV assays. Target primer concentrations for IS1 and 23s rDNA in both assays were then tested from 100 to 700 nM in multiplex reactions with the CMV primer-probe set against prepared ratio mixtures of pIS1 or p23sTA and V1Jns-tpa-gag (CMV promoter target). 500 nM of the IS1-Q primers provided the most accurate results in multiplex with CMV-Q primers when compared to copy ratios determined from IS1- and CMV-only reactions, while 200 nM of 23s rDNA primers provided equivalent accuracy in the 23s rDNA/CMV multiplex reactions (data not shown).

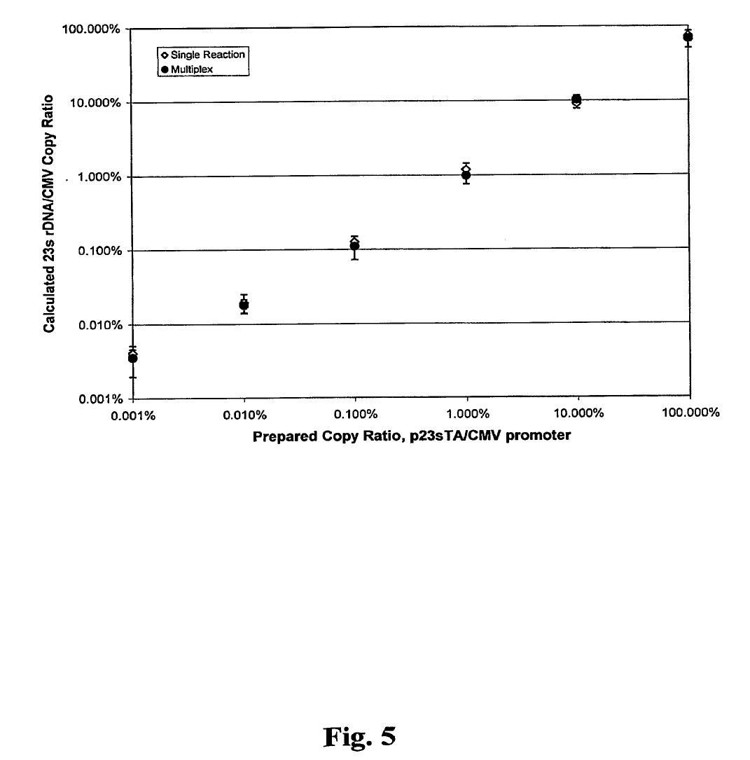

Q-PCR assay sensitivity—Sensitivity studies were performed on both the IS1/CMV and 23s rDNA/CMV multiplex Q-PCR assays. To determine the point at which spectral interference significantly inhibits 23s rDNA quantitation relative to plasmid DNA, it is necessary to use ratios of 23s rDNA to CMV fragments over a wide range for analysis with the 23s rDNA/CMV Q-PCR copy ratio assay. Residual genomic DNA in plasmid preparations limits the range over which ratios can be tested if a plasmid is the source of the CMV promoter template. Therefore, the CMV promoter region of plasmid V1Jns-tpa-gag was PCR-amplified (primers 5′-CACTGTTAGGAGCAAGGAGC-3′ (SEQ ID NO: 14) and 5′-TGACGACTGAATCCGGTGAG-3′ (SEQ ID NO:15)), decreasing the 23s rDNA/CMV ratio to ≦1.7×10⁻⁵. The amplified CMV promoter fragment was then used as the CMV template to prepare 1:1 to 1:10⁵ 23s rDNA/CMV ratio samples. Single and multiplex (CMV primer-limited) reaction results for all samples tested were equivalent, and the response was linear over 5 orders of magnitude (FIG. 5). The sensitivity of the IS1/CMV assay was tested in a similar fashion, using mixtures of 103-10 copies pIS1/μL combined with 10⁸ copies CMV promoter fragment/μL, and resulted in equivalent results for both single and multiplex reaction results for all ratio samples. Hence, the assay was qualified to a limit of quantitation of 1:10⁵ (0.001%) copies IS1 or 23s rDNA per copy CMV.

Q-PCR assay sensitivity vs. agarose gel electrophoresis—The IS1 transposon was originally observed in experimental samples of plasmid V1Jns-nef isolated from cultures that had been shifted from complex to defined medium and cultured for ˜30 generations (see Example 1). Analysis of plasmid DNA from these 10 clones (samples NLB-1 through NLB-10) by agarose gel electrophoresis indicated that the transposon was present based on the appearance of a high molecular weight band in at least five clones (starred lanes in FIG. 1B; see also Example 1). All 10 NLB samples were analyzed with the IS1/CMV copy ratio assay (see Table 1). Seven of the samples contained ≧1% IS1-positive plasmid DNA. The five samples with visible transposons contained ≧5% IS1-positive molecules. These results suggest that on the order of 5-10% of plasmid DNA must be altered to visualize the anomalies with ethidium bromide staining of agarose gels.

TABLE 1
IS1-positive plasmid DNA fractions as determined
from the IS1/CMV copy ratio assay.

| | Copy Ratio | |
|---|---|---|
| Sample | (IS1/CMV) | ±Std. Dev. |
| NLB-1 | 7.0% | 0.6% |
| NLB-2 | 0.7% | 0.13% |
| NLB-3 | 7.8% | 0.8% |
| NLB-4 | 0.4% | 0.05% |
| NLB-5 | 16.0% | 1.6% |
| NLB-6 | 1.0% | 0.1% |
| NLB-7 | 14.6% | 3.1% |
| NLB-8 | 10.9% | 1.3% |
| NLB-9 | 0.7% | 0.08% |
| NLB-10 | 1.0% | 0.1% |

Summary—The IS1/CMV Q-PCR copy ratio assay is a valuable tool in the characterization of DNA vaccine clones. The assay is highly specific. It can easily distinguish between samples containing the IS1 transposon, samples containing other transposons such as IS5, and transposon-negative samples through its reliance on specially designed oligonucleotide primers and fluorescent probes. The inclusion of the 23s rDNA/CMV copy ratio assay further increases the specificity, allowing for precise quantitation of increases in IS1 transposition activity. The high level of sensitivity offered by the Q-PCR technology allows for the quantitation of IS1 transposition over six logs of template DNA concentration while detecting targets at concentrations at least as low as 100 copies per μL (0.6 pg/ml for V1Jns-nef).

### EXAMPLE 5

### Identification of IS1 Insertational Mutations in Genomic DNA

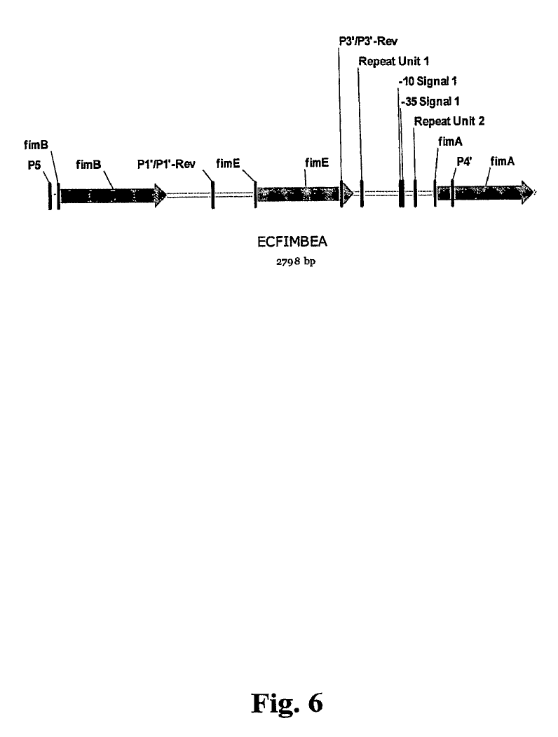

A prior screening methodology for isolation of potentially highly productive clones is based on differences in colony morphology between “Gray” and “White” clones as they appear on Columbia Blood Agar plates after incubation at 28-30° C. for approximately 48 hours (see co-pending International Application No. PCT/US2005/002911, supra). Insertion sequence mutations in genes related to fimbriae formation are known to affect colony morphology (La Ragione et al., 1999, FEMS Microbiol Lett. 175:247-253; Stentebjerg-Olson et al., 2000, FEMS Microbiol. Lett. 182:319-325). Additionally, a phase-switch caused by an inversion of a region of the regulatory sequence of the fimA gene also leads to differences in the expression of fimbriae (Stentebjerg-Olesen et al., 2000, supra). Using PCR, the presence of insertion elements in the fimBEA operon and the csgB gene were investigated in naïee ve and DME-P5-adapted DH5 cells at both 28-30° C. and 37° C. The adapted cells display a morphological switch between 28-30° C. and 37° C. on blood agar plates. Differences in the fimbriae genes could therefore be correlated to this switch. Using genomic DNA isolated from shake flask cultures of both cells at 37° C. and static (blood agar-grown) cultures at both temperatures, no differences in either the fimA phase switch or insertion elements were observed among the samples. The representative high- and low-producers, tpa-gag-gray and tpa-gag-white, respectively, were also profiled with no differences observed. Interestingly, an IS1 insertion was identified based on restriction digests in the amplified region that includes most of the fimE gene and upstream sequences (FIG. 6, between region P1′-P3′). This site is not reported in the published E. coli sequences of the non-pathogenic K-12 strains MG1655 or W3110 available on GenBank. Based on the surrounding AflII and AgeI restriction sites, the fragment containing this IS1 insertion would be either 2.3 Kb or ˜1.7 Kb in length. The latter fragment size is consistent with the smallest band in the RFLP profiles of both transformed and plasmid-free strains (see Example 2). Because the insertion was found in all samples and the affected fragment generated equivalent bands upon further digestion with IS1-specific enzymes, it is unlikely that the insertion sequence contributes to the observed differences in colony morphology.

Construction of the pMCS2 cloning vector—A pUC19-based vector was constructed that confers resistance to neomycin (pUC-neo). The amp^{R} gene (bla) of pUC19 (New England Biolabs) was replaced by the kan^{R}/neo^{R} gene (nptII) taken from the pUC-4K plasmid (Amersham Pharmacia Biotech). The amp^{R} gene in pUC19 was removed by digestion with restriction enzymes AhdI (Eam 1105I) and SspI, and the neo^{R} gene was removed from the pUC-4K plasmid by digestion with the restriction enzyme PstI. Both fragments were made blunt-ended using the Klenow fragment of E. coli DNA Polymerase. The 10.8 kb pUC19 fragment containing the replication machinery and the 1.2 kb fragment containing the nptII gene from pUC4K were purified by agarose gel electrophoresis and then ligated with T4 DNA ligase. Resulting plasmids were screened to identify those with nptII insertions in the same orientation as the original bla gene to complete construction of pUC-neo.

To construct pMCS2, restriction sites for EcoRI, AflI, and BamHI (5′→3′) were incorporated into the 5′ end of a forward primer to amplify the hisC gene from E. coli (MCS2-hisC-For: 5′-GAATTCTTAAGATAGGATCCAAGGAGCAAGCATGAGCACC-3′; SEQ ID NO:16). The hisC gene was arbitrarily chosen for this purpose. Sites for XbaI, AgeI, and BamHI (5′→3′) were similarly incorporated into the reverse primer (MCS2-hisC-Rev: 5′-TCTAGACCGGTATGGATCCCGCGATCGATAAAAAGATAC-3′; SEQ ID NO:17). PCR amplification was used to generate a 1.1 Kb fragment consisting of the new multi-cloning site, containing AflII and AgeI, with the intervening hisC gene between BamHI sites. The hisC gene was included to provide a large fragment for ligation, and the BamHI sites were used to remove this gene and restore the lacZα ORF for blue/white selection. The resulting fragment was ligated to the pCR2.1-TOPO vector (Invitrogen, Carlsbad, Calif.). The 1.1 Kb EcoRI-XbaI fragment was excised and successfully ligated to pUC-neo. The resulting plasmid, pMCS2-hisC, was digested with BamHI to remove the hisC gene, and the largest (3.1 Kb) fragment was gel-extracted and re-ligated to form plasmid pMCS2. As mentioned, the pMCS2 multi-cloning site was designed to preserve the open reading frame (ORF) of the lacZα gene and retain blue-white selection. Transformants of the pMCS2 ligation were recovered on LB/neo agar supplemented with IPTG and X-gal, and the resulting colonies were blue, indicating that the lacZ ORF had been retained. Vector pMCS2 was then partially sequenced to confirm the structure of the multi-cloning site.

Screening for the IS insertion site—RFLP analysis indicated that the insertional mutation of interest lies on a restriction fragment between 3 and 4 Kb when the genomic DNA is digested with the restriction enzymes AflII and AgeI (see Example 2). A strategy to identify the site(s) of the insertional mutations in the genomic DNA consists of assembling and screening a library of genomic DNA fragments from the affected clones according to the following steps:

1. Extraction of the genomic DNA from identified low-producer clones exhibiting the IS1 insertional mutation. Depending on the methods chosen, extraction of the genomic DNA may result in a mixture that includes plasmid DNA.

2. Digestion of the DNA with AflII and AgeI to produce restriction fragments containing IS1 of the previously identified size, ˜3-4 Kb.

3. Extraction of the desired fragment sizes from the pool of restriction fragments. This may be accomplished, for example, through gel electrophoresis for separation of the fragments based on size, followed by excision of a portion of the gel containing fragments of the desired range, and isolation of the fragments from the excised gel. If plasmid DNA is retained in the original genomic DNA preparation, the desired genomic DNA fragments should be isolated from the mixture to remove the high background associated with re-ligation and transformation of the linearized plasmid DNA.

4. Ligation of the pool of desired fragments into a vector linearized with restriction enzymes AflII and AgeI in a multi-cloning site. Such a vector, pMCS2, was created for this purpose (see above). The ligated pool can be used to transform competent E. coli cells, and the transformants can be propagated on solid LB or other complex medium suitable for rapid growth and maintenance.

5. Screening of the resulting library can be accomplished in several ways, including, but not limited to: (a) use of a Q-PCR assay specific for IS1; and, (b) colony or plasmid PCR specific to IS1:plasmid junctions. These potential screening methods are described in more detail below.

Q-PCR assay specific for IS1 in genomic DNA library—The vector pMCS2 created for constructing a genomic DNA library, described above, contains a gene for neomycin resistance (neo^{R}). Plasmids in the library consisting of an IS1-positive genomic DNA fragment ligated with the pMCS2 vector will contain IS1 and neo^{R} in a 1:1 ratio. IS1-negative plasmids should contain only background amounts of IS1 from residual genomic DNA. The fraction of IS1 contributed by genomic DNA should be small compared to the amount contributed by plasmid DNA since pMCS2 is a high-copy number plasmid. Therefore, a Q-PCR assay similar to that described in Example 4 can be performed with purified plasmid DNA or whole cells. A Q-PCR assay is contemplated to determine IS1:neo^{R} copy ratios in multiplex mode using the following primer/probe sequences:

IS1 primer/probe set:
IS1-Q-For:

(SEQ ID NO:18)

5′-AGGCTCATAAGACGCCCCA-3′;
IS1-Q-Rev:

(SEQ ID NO:19)

5′-ACGGTTGTTGGGCACGTAT-3′;
and,
IS1-Q-Probe:

(SEQ ID NO:20)

5′-VIC-CGTCGCCATAGTGCGTTCACCG-TAMRA-3′.
neo^{R} primer/probe set:
neo-Q-For:

(SEQ ID NO:21)

5′-CAACCTATTAATTTCCCCTCGTCA-3′;
neo-Q-Rev:

(SEQ ID NO:22)

5′-CTGGCCTGTTGAACAAGTCTG-3′;
and,
neo-Q-Probe:

(SEQ ID NO:23)

5′-FAM-CCATGAGTGACGACTGAATCCGGTG-TAMRA-3′.

Initial multiplex optimization experiments were performed to identify optimal primer concentrations of 100 nM for the IS1-Q primers and 300 nM for the neo-Q primers, with 200 nM for both IS1 and neo probes. These conditions were determined by limiting the IS1-Q primer concentrations to 100 nM while testing neo-Q primer concentrations at a range of between 100 nM-400 nM. IS1-Q had been shown to negatively affect neo-Q throughout multiplex primer optimization experiments, especially when limiting neo-Q primer concentrations. With the newly optimized values, IS1-Q spectral interference still alters neo-Q threshold cycle (C_{T}) values by up to 70% at 0.1% IS1/neo copy ratios, but the results are well within the tolerance required for the screen.

Colony and plasmid PCR specific to IS1 plasmid junctions—Since both whole cells and purified plasmid DNA are expected to contain genomic DNA, PCR specific to IS1 is expected to result in amplification from all samples, whether an IS1-positive genomic DNA fragment is present in the plasmid library or not. However, PCR using one primer specific to the plasmid and one primer specific to IS1 should only produce a signal if the recombinant plasmid contains an IS1 fragment. The plasmid-specific primer can be designed in several ways. It could anneal to a region of the plasmid near the insertion site (i.e., the AflII-AgeI recognition sites) to avoid an unfavorably large amplicon size of up to 4 Kb if the transposon is near the opposite end of the fragment. In this case, polymerases that are favorably disposed to amplification of long targets could be used to ensure satisfactory amplification. Alternatively, primers could anneal to a region removed from the insertion site on either side to avoid amplification of unusually small fragments. In either case, it is important than an assay utilizing PCR to identify IS1 plasmid junctions account for the possibility of different IS1 orientations in designing the primers.

To address this issue, two IS1-specific primers complementary to the center region of the transposon but extending in either direction could be employed to ensure that both orientations of the transposon insertion would be detected. Use of an internal primer also ensures the amplification of a target that is at least 380 bp. Since this screening assay is specific for IS1:plasmid junctions, it can be performed with whole cells (“colony PCR”) or purified plasmid DNA. It is not expected that IS1 present in the genomic DNA would return a signal.

### EXAMPLE 6

### Genome-Based High Throughput Screen for Highly Productive Clones

The RFLP profiles disclosed in Example 2 revealed a correlation between low-producing DNA vaccine clones and IS1 insertional mutations in the genomic DNA. A high throughput screen for highly-productive clones would therefore consist of the identification and selection of clones that do not carry the insertional mutation. Such a screen would require the identification of the mutation/insertion sites, for example, as described in Example 5. Several assays could subsequently be developed for this screening process.

TaqMan Q-PCR-based high throughput screen—One example of a TaqMan Q-PCR high throughput assay to identify bacterial clones that do not carry the IS1 insertion mutation requires the use of two primers and an internal probe for amplification of a fluorescent signal (diagramed in FIG. 7A). If the IS1 insertion mutation is localized to a single, identified site within the genomic DNA, the TaqMan probe can be designed to recognize this part of the genome. Amplification utilizing the complementary primers would result in accumulation of a fluorescent signal due to degradation of the probe as described previously (see Example 4). Presence of an IS1 insertion at the site in question would disrupt the binding site and prevent the accumulation of fluorescence. Thus, a potential high-producer clone would give an amplification signal whereas a low-producer would exhibit only background fluorescence due to the inherent noise of the system. The assay does not require multiplexing and can be performed using a whole cells lysate, eliminating the need for isolation of the genomic DNA. The TagMan probes typically vary in length from about 15 to about 40 nucleotides. Therefore, the identified hotspot for the IS1 mutation must be localized to a narrow range of sequences, preferably within 10 nucleotides to ensure adequate binding of the genome-specific probe.

PCR assay for IS1:genome junctions—If the insertion site is not localized to a very narrow region of the genomic DNA, a PCR assay that does not use internal probes can be employed to identify IS1:genomic DNA junctions (diagramed in FIG. 7B). One primer can be designed to anneal to the genomic DNA a short distance removed from the IS1 insertion hotpot. The second primer should anneal to the transposon. If the insertion is present (i.e., a putative “low-producer”), an amplified fragment corresponding to the IS1:genomic DNA junction should be produced. The resulting amplification products can be analyzed visually to identify fragments of the target size. Alternatively, a dye such as SYBR® Green can be added to the assay and a real-time PCR instrument can be used to identify potential highly-productive clones based on the corresponding increase in fluorescence (e.g., a lack of fluorescence indicates a lack of IS1 insertion—a putative “high-producer”). Similarly, a fluorogenic LUX™ primer (Invitrogen) could be employed to measure exponential increases in fluorescence. Note that in this case, a fluorescent signal is expected even in clones without the genome:IS1 junction since the signal from a LUX primer arises from extension of a single primer. However, the signal would increase linearly and not exponentially because of the absence of a complementary primer to produce a single amplified fragment.

To avoid the need to analyze amplified fragments visually, the identified insertion mutation must be well-removed from the 7 static copies of IS1 in the genome to prevent false positives. Based on the RFLP profiles disclosed in Example 2, it does not appear as if the static copies are sufficiently close to the mutation to cause interference. This assay must also account for the possibility of either orientation of insertion of the mutation. This can be done by using internal IS1 primers in either orientation. In this case, two separate assays could be run per sample or both primers could be utilized simultaneously to completely screen the population of clones.

### EXAMPLE 7

### Construction of Optimized Strains for DNA Vaccine Production

Because the low-producer population is correlated with an IS1 insertional mutation, it is conceivable that an E. coli host strain devoid of any IS1 copies would result in a more uniform population of highly-productive clones. Therefore, one strategy for improving the yield of highly productive clones involves constructing a strain of E. coli in which all of the IS1 copies have been removed, and using said strain for the propagation of DNA vaccine vectors. Several methods exist for the construction of deletion or disruption mutations of E. coli including P1 phage transduction, transposon-mediated random mutagenesis, and generalized (RecA-mediated) homologous recombination. These methods are typically suitable for single mutations but not multiple ones due to the need for a selectable marker, for example, antibiotic resistance, for each mutation. An alternative method involves the use of PCR products with 36- to 50-nt extensions on the primers that are homologous to the flanking sequences around the desired disruption site, and the lambda-Red recombinase (Datsenko and Wanner, 2000, PNAS 97:6640-6645). A selectable marker is still used in this case; however, the marker can be subsequently removed, freeing its use for additional rounds of mutation. A modified method that eliminates residual “scars” utilizes the endogenous double-strand break repair process to remove the selectable marker (Kolisnychenko et a., 2002, Genome Res. 12:640-647). This method was used to produce a K-12 strain of E. coli with an 8.1% reduction in genome size, including elimination of 24 of 44 transposable elements. Three of the seven IS1 copies were removed in this strain. It is highly probable that removal of the remaining 4 copies will have no deleterious effects on the survivability of the strain or suitability of its use in the fed-batch fermentation process. Note, however, that the modified method of Kolisnychenko et al. is not suitable for E. coli strain DH5 due to the need for RecA in the double-strand break repair process. Another method utilizes group II introns, so-called “targetrons,” to produce mutations based on 14- to 16-nt regions of complementary sequence (Zhong et al., 2003, Nucleic Acids Res. 31:1656-1664). This method also utilizes a selectable marker than can be subsequently removed to allow for multiple insertions. However, it does not produce deletions of the target sites as the two previous methods, but rather produces disruptions. Use of this method would result in a strain that carries 7 non-functional copies of IS1, being disrupted in the main transposase gene (insAB) encoded by the transposon.

1. A method for selecting a highly productive clonal subtype of a strain of E. coli harboring a plasmid DNA comprising:
(a) comparing IS1 transposition activity in at least two clonal subtypes of the same strain harboring the same plasmid DNA, wherein the clonal subtype that displays a comparatively lower transposition activity represents a potential highly productive clonal subtype; and,
(b) testing productivity of said potential highly productive clonal subtype;
wherein a highly productive clonal subtype exhibits a high plasmid copy number per cell.

2. A method of claim 1, wherein IS1 transposition activity is determined by measuring IS1 transposon copy number in isolated plasmid DNA samples from said clonal subtypes, wherein a comparatively lower IS1 transposon copy number indicates a comparatively lower IS1 transposition activity.

3. A method of claim 1, wherein IS1 transposition activity is determined by measuring the presence or absence of an IS1 transposon sequence in a predetermined IS1 insertion region within genomic DNA of said clonal subtypes, wherein the absence of an IS1 insertion sequence indicates a comparatively lower IS1 transposition activity.

4. A method for selecting a highly productive clonal subtype of a strain of E. coli harboring a plasmid DNA comprising:
(a) isolating plasmid DNA from at least two clonal subtypes of the same strain harboring the same plasmid DNA;
(b) measuring IS1 transposon copy number in said isolated plasmid DNA samples, wherein the clonal subtype that displays a comparatively lower IS1 transposon copy number represents a potential highly productive clonal subtype; and
(c) testing productivity of said potential highly productive clonal subtype; wherein a highly productive clonal subtype exhibits a high plasmid copy number per cell.

5. A method of claim 4, wherein the IS1 transposon copy number is measured using a quantitative PCR assay.

6. A method of claim 5, wherein the quantitative PCR assay measures the relative quantity of IS1 based on plasmid copy number by amplifying both a first nucleotide sequence of the plasmid DNA located within the IS1 nucleotide sequence and a second nucleotide sequence of the plasmid DNA determined to be free of IS1 insertions, generating an IS1/plasmid copy ratio which represents the IS1 transposon copy number.

7. A method of claim 6, wherein the IS1/plasmid copy ratio is corrected by subtracting the predicted quantity of IS1 transposon copies contributed from residual genomic DNA present in the plasmid DNA sample.

8. A method of claim 7, wherein the predicted quantity of IS1 transposon copies contributed from residual genomic DNA present in the plasmid DNA sample is measured using a second quantitative PCR assay.

9. A method of claim 8, wherein the second quantitative PCR assay measures the relative quantity of 23s rDNA based on plasmid copy number by amplifying both a nucleotide sequence of the residual genomic DNA located within the 23s rDNA nucleotide sequence and the second nucleotide sequence of the plasmid DNA determined to be free of IS1 insertions used to generate the IS1/plasmid copy ratio, generating a 23s rDNA/plasmid copy ratio that is subtracted from the IS1/plasmid copy ratio to provide a corrected IS1/plasmid copy ratio.

10. A method of claim 6, wherein the first and second nucleotide sequences of the plasmid DNA are individually amplified in the presence of a nucleic acid polymerase and a set of oligonucleotides, wherein the set of oligonucleotides used to amplify the first nucleotide sequence consists of:
(i) a forward PCR primer that hybridizes to a first location of the IS1 nucleotide sequence;
(ii) a reverse PCR primer that hybridizes to a second location of the IS1 nucleotide sequence downstream of the first location; and,
(iii) a fluorescent probe labeled with a quencher molecule and a fluorophore which emits energy at a unique emission maxima, said probe hybridizes to a location within the IS1 nucleotide sequence between the first and second locations;
and the set of oligonucleotides used to amplify the second nucleotide sequence consists of:
(i) a forward PCR primer that hybridizes to a first location of the second nucleotide sequence;
(ii) a reverse PCR primer that hybridizes to a second location of the second nucleotide sequence downstream of the first location; and
(iii) a fluorescent probe labeled with a quencher molecule and a fluorophore which emits energy at a unique emission maxima, said probe hybridizes to a location within the second nucleotide sequence between the first and second locations;
wherein said nucleic acid polymerase digests the fluorescent probes during amplification to dissociate said fluorophores from said quencher molecules, and a change of fluorescence upon dissociation of the fluorophore and quencher molecules is detected, the change of fluorescence corresponding to the occurrence of amplification of the first and/or second nucleotide sequences.

11. (canceled)

12. A method of claim 10, wherein the set of oligonucleotides used to amplify the first nucleotide sequence consists of forward and reverse PCR primers IS1-Q-F (SEQ ID NO:6) and IS1-Q-R (SEQ ID NO:7), respectively, and fluorescent probe IS1-Q-P2 (SEQ ID NO:8); and the set of oligonucleotides used to amplify the second nucleotide sequence consists of forward and reverse PCR primers CMV-Q-F (SEQ ID NO:3) and CMV-Q-R (SEQ ID NO:4), respectively, and fluorescent probe CMV-Q-P2 (SEQ ID NO:5).

13. (canceled)

14. A method of claim 9, wherein the nucleotide sequence located within the 23s rDNA sequence and the second nucleotide sequence are individually amplified in the presence of a nucleic acid polymerase and a set of oligonucleotides, wherein the set of oligonucleotides used to amplify the first nucleotide sequence consists of:
(i) a forward PCR primer that hybridizes to a first location of the 23s rDNA sequence;
(ii) a reverse PCR primer that hybridizes to a second location of the 23s rDNA sequence downstream of the first location; and,
(iii) a fluorescent probe labeled with a quencher molecule and a fluorophore which emits energy at a unique emission maxima; said probe hybridizes to a location within the 23s rDNA sequence between the first and second locations;
and the set of oligonucleotides used to amplify the second nucleotide sequence consists of:
(iv) a forward PCR primer that hybridizes to a first location of the second nucleotide sequence;
(v) a reverse PCR primer that hybridizes to a second location of the second nucleotide sequence downstream of the first location; and
(vi) a fluorescent probe labeled with a quencher molecule and a fluorophore which emits energy at a unique emission maxima, said probe hybridizes to a location within the second nucleotide sequence between the first and second locations;
wherein said nucleic acid polymerase digests the fluorescent probes during amplification to dissociate said fluorophores from said quencher molecules, and a change of fluorescence upon dissociation of the fluorophore and quencher molecules is detected, the change of fluorescence corresponding to the occurrence of amplification of the 23s rDNA sequence and/or second nucleotide sequence.

15. (canceled)

16. A method of claim 14, wherein the set of oligonucleotides used to amplify the 23s rDNA nucleotide sequence consists of forward and reverse PCRprimers 23s-F1D (SEQ ID NO:11) and 23s-RID (SEQ ID NO:12), respectively, and fluorescent probe 23s-Pfam (SEQ ID NO: 13); and the set of oligonucleotides used to amplify the second nucleotide sequence consists of forward and reverse PCR primers CMV-Q-F (SEQ ID NO:3) and CMV-Q-R (SEQ ID NO:4), respectively, and fluorescent probe CMV-Q-P2 (SEQ ID NO:5).

17. (canceled)

18. A method for selecting a highly productive clonal subtype of a strain of E. coli harboring a plasmid DNA comprising:
(a) detecting the presence or absence of an IS1 transposon sequence within a predetermined IS1 insertion region of the genomic DNA of said clonal subtype, wherein a clonal subtype lacking an IS1 transposon sequence within said IS1 insertion region represents a potential highly productive clonal subtype; and,
(b) testing productivity of said potential highly productive clonal subtype; wherein a highly productive clonal subtype exhibits a high plasmid copy number per cell.

19. A method of claim 18, wherein said IS1 insertion region spans less than about 20 contiguous nucleotides of the genomic DNA.

20. A method of claim 19, wherein a quantitative PCR assay is used to detect the presence or absence of an IS1 transposon sequence within said IS1 insertion region of the genomic DNA.

21. A method of claim 20, wherein the quantitative PCR assay amplifies a portion of the genomic DNA that contains the IS1 insertion region in the presence of a nucleic acid polymerase and a set of oligonucleotides consisting of:
(i) a fluorescent probe labeled with a quencher molecule and a fluorophore which emits energy at a unique emission maxima, wherein said probe hybridizes to a location within the genomic DNA that spans the IS1 insertion region only when said genomic DNA lacks an IS1 transposon sequence within said IS1 insertion region;
(ii) a forward PCR primer that hybridizes to a location of the genomic DNA upstream of the fluorescent probe; and,
(iii) a reverse PCR primer that hybridizes to a location of the genomic DNA downstream of the fluorescent probe;
wherein said nucleic acid polymerase digests the fluorescent probe during amplification to dissociate said fluorophore from said quencher molecule, and a change of fluorescence upon dissociation of the fluorophore and the quencher molecule is detected, the change of fluorescence corresponding to amplification of the genomic DNA and the absence of an IS1 transposon sequence within the IS1 insertion region.

22. A method of claim 21, wherein said quantitative PCR assay is performed on a whole cell lysate.

23. A method of claim 18, wherein said IS1 insertion region spans greater than about 20 contiguous nucleotides of the genomic DNA.

24. A method of claim 23, wherein a PCR assay is used to detect the presence or absence of an IS1 transposon sequence within said IS1 insertion region of the genomic DNA.

25. A method of claim 24, wherein the PCR assay amplifies a portion of the genomic DNA in the presence of a nucleic acid polymerase and a set of oligonucleotides consisting of:
(i) a first PCR primer that hybridizes to a location of the genomic DNA outside of the IS1 insertion region; and,
(ii) a second PCR primer that hybridizes to a location within an IS1 transposon sequence inserted within the IS1 insertion region;
wherein the presence of an IS1 transposon sequence within the IS1 insertion region results in exponential amplification of said portion of the genomic DNA due to the hybridization of both PCR primers, and the absence of an IS1 transposon sequence within the IS1 insertion region results in linear amplification of only a single strand of said portion of the genomic DNA due to hybridization of only the first PCR primer.

26. A method of claim 25, wherein amplification of said portion of the genomic DNA is visually detected by identifying amplified nucleic acid fragments of approximate target size.

27. A method of claim 25, wherein amplification of said portion of the genomic DNA is fluorescently detected in real-time by adding a nucleic acid stain that binds double-stranded DNA.


SEQUENCE LISTING (23 sequences)

SEQ ID NO: 1 - DNA, 21 bp, Artificial Sequence
  oligonucleotide
ggtaatgactccaacttattg

SEQ ID NO: 2 - DNA, 18 bp, Artificial Sequence
  oligonucleotide
ggtgatgctgccaactta

SEQ ID NO: 3 - DNA, 25 bp, Artificial Sequence
  oligonucleotide
gtacggtgggaggtctatataagca

SEQ ID NO: 4 - DNA, 21 bp, Artificial Sequence
  oligonucleotide
ggaggtcaaaacagcgtggat

SEQ ID NO: 5 - DNA, 27 bp, Artificial Sequence
  oligonucleotide
tcgtttagtgaaccgtcagatcgcctg

SEQ ID NO: 6 - DNA, 19 bp, Artificial Sequence
  oligonucleotide
aggctcataagacgcccca

SEQ ID NO: 7 - DNA, 19 bp, Artificial Sequence
  oligonucleotide
acggttgttgcgcacgtat

SEQ ID NO: 8 - DNA, 22 bp, Artificial Sequence
  oligonucleotide
cgtcgccatagtgcgttcaccg

SEQ ID NO: 9 - DNA, 26 bp, Artificial Sequence
  oligonucleotide
ggatccaacccagtgtgtttcgacac

SEQ ID NO: 10 - DNA, 26 bp, Artificial Sequence
  oligonucleotide
ggatccagacaggataccacgtgtcc

SEQ ID NO: 11 - DNA, 18 bp, Artificial Sequence
  oligonucleotide
gaaaggcgcgcgatacag

SEQ ID NO: 12 - DNA, 19 bp, Artificial Sequence
  oligonucleotide
gtcccgccctactcatcga

SEQ ID NO: 13 - DNA, 26 bp, Artificial Sequence
  oligonucleotide
ccccgtacacaaaaatgcacatgctg

SEQ ID NO: 14 - DNA, 20 bp, Artificial Sequence
  oligonucleotide
cactgttaggagcaaggagc

SEQ ID NO: 15 - DNA, 20 bp, Artificial Sequence
  oligonucleotide
tgacgactgaatccggtgag

SEQ ID NO: 16 - DNA, 40 bp, Artificial Sequence
  oligonucleotide
gaattcttaagataggatccaaggagcaagcatgagcacc

SEQ ID NO: 17 - DNA, 39 bp, Artificial Sequence
  oligonucleotide
tctagaccggtatggatcccgcgatcgataaaaagatac

SEQ ID NO: 18 - DNA, 19 bp, Artificial Sequence
  oligonucleotide
aggctcataagacgcccca

SEQ ID NO: 19 - DNA, 19 bp, Artificial Sequence
  oligonucleotide
acggttgttgcgcacgtat

SEQ ID NO: 20 - DNA, 22 bp, Artificial Sequence
  oligonucleotide
cgtcgccatagtgcgttcaccg

SEQ ID NO: 21 - DNA, 24 bp, Artificial Sequence
  oligonucleotide
caacctattaatttcccctcgtca

SEQ ID NO: 22 - DNA, 21 bp, Artificial Sequence
  oligonucleotide
ctggcctgttgaacaagtctg

SEQ ID NO: 23 - DNA, 25 bp, Artificial Sequence
  oligonucleotide
ccatgagtgacgactgaatccggtg